(19) United States
(12) Patent Application Publication
(10) Pub. No.: US 2017/0246473 A1
(43) Pub. Date: Aug. 31, 2017

(54) METHOD AND SYSTEM FOR MANAGING TREATMENTS

(71) Applicants: SAVA MARINKOVICH, JERSEY CITY, NJ (US); RAHUL MEHENDALE, JERSEY CITY, NJ (US)

(72) Inventors: SAVA MARINKOVICH, JERSEY CITY, NJ (US); RAHUL MEHENDALE, JERSEY CITY, NJ (US)

(21) Appl. No.: 15/053,041

(22) Filed: Feb. 25, 2016

(51) Int. Cl. A61N 5/06; G01N 21/84; G01N 21/25; G01N 21/55; G06F 19/00; A61B 5/00

(52) U.S. Cl. CPC A61N 5/0616; G06F 19/321; G06F 19/345; G06F 19/327; G06F 19/3431; A61B 5/445; A61B 5/0059; A61B 5/4848; G01N 21/255; G01N 21/55; G01N 21/84; A61N 2005/0663; A61N 2005/0626

(57) ABSTRACT

Embodiment of the present invention disclose a method of managing treatment comprising capturing images of an organ, analyzing the captured images, determining at least one of susceptibility to at least one of a medical and non-medical condition and presence of the at least one of medical and non-medical condition, upon determination of the presence of at least one of medical and non-medical condition, measuring the level of severity of the at least one of medical and non-medical condition, categorizing the at least one of medical and non-medical condition based on the measured level of severity, recommending one or more of at least one of preventive, palliative, curative and cosmetic cum aesthetic, and at least one of new and enhanced personalized treatments based on the measured level of severity of the at least one of medical and non-medical condition, implementing the one or more of at least one of preventive, palliative, curative and cosmetic cum aesthetic, new and enhanced personalized treatments, tracking efficacy of the implemented one or more of at least one of preventive, palliative, curative and cosmetic cum aesthetic, new and enhanced personalized treatments and optimizing the one or more of at least one of preventive, palliative, curative and cosmetic cum aesthetic, new and enhanced personalized treatments based on the efficacies thereof.

### CROSS-REFERENCE TO RELATED APPLICATIONS

This application claims benefit of the following U.S. Provisional Application, which is incorporated herein by reference in its entirety: U.S. Provisional Patent Application No. 62/120,378 filed Feb. 25, 2015.

This application is a continuation-in-part of the following U.S. Non-Provisional application, which is hereby incorporated by reference in its entirety: U.S. Non-Provisional patent application Ser. No. 14/799,687, filed Jul. 15, 2015, which claims benefit of the of the following U.S. Provisional Applications, each of which is incorporated herein by reference in its entirety: U.S. Provisional Patent Application No. 62/024,699 filed Jul. 15, 2014 and U.S. Provisional Patent Application No. 62/120,378 filed Feb. 25, 2015, and which in turn is a continuation-in-part of the following U.S. Non-Provisional application, which is hereby incorporated by reference in its entirety: U.S. Non-Provisional patent application Ser. No. 11/970,448, filed Jan. 7, 2008, and which claims the benefit of the following U.S. Provisional Applications, each of which is hereby incorporated by reference in their entirety: U.S. Provisional Patent Application No. 60/883,769, filed Jan. 5, 2007; U.S. Provisional Patent Application No. 60/883,764, filed Jan. 5, 2007; and U.S. Provisional Patent Application No. 60/883,768, filed Jan. 5, 2007.

This application is a continuation-in-part of the following U.S. Non-Provisional application, which is hereby incorporated by reference in its entirety: U.S. Non-Provisional patent application Ser. No. 14/799,687, filed Jul. 15, 2015, which in turn is a continuation-in-part application of the following U.S. Non-Provisional application, which is hereby incorporated by reference in its entirety: U.S. Non-Provisional patent application Ser. No. 12/350,164, filed Jan. 7, 2009, and which claims the benefit of the following U.S. Provisional Applications, each of which is hereby incorporated by reference in their entirety: U.S. Provisional Patent Application No. 61/019,440, filed Jan. 7, 2008 and U.S. Provisional Patent Application No. 61/061,852, filed Jun. 16, 2008.

This application is a continuation-in-part of the following U.S. Non-Provisional application, which is hereby incorporated by reference in its entirety: U.S. Non-Provisional patent application Ser. No. 14/799,687, filed Jul. 15, 2015, which in turn is a continuation-in-part of U.S. Non-Provisional patent application Ser. No. 12/690,749, filed Jan. 20, 2010, which is incorporated herein by reference in its entirety and which claims the benefit of the following U.S. Provisional applications, each of which is hereby incorporated by reference in its entirety: U.S. Provisional Patent Application No. 61/145,756, filed Jan. 20, 2009; U.S. Provisional Patent Application No. 61/150,010, filed Feb. 5, 2009; U.S. Provisional Patent Application No. 61/149,025, filed Feb. 2, 2009; U.S. Provisional Patent Application No. 61/149,027, filed Feb. 2, 2009; U.S. Provisional Patent Application No. 61/150,053, filed Feb. 5, 2009; U.S. Provisional Patent Application No. 61/150,331, filed Feb. 6, 2009; U.S. Provisional Patent Application No. 61/169,316, filed Apr. 15, 2009; U.S. Provisional Patent Application No. 61/235,362, filed Aug. 20, 2009; and U.S. Provisional Patent Application No. 61/254,214, filed Oct. 23, 2009.

This application is a continuation-in-part of the following U.S. Non-Provisional application, which is hereby incorporated by reference in its entirety: U.S. Non-Provisional patent application Ser. No. 14/799,687, filed Jul. 15, 2015, which in turn is a continuation-in-part of U.S. Non-Provisional patent application Ser. No. 13/036,783 filed Feb. 28, 2011, which is incorporated herein by reference in its entirety and which claims the benefit of the following U.S. Provisional Applications, each of which is hereby incorporated by reference in its entirety: U.S. Provisional Patent Application No. 61/310,287, filed Mar. 4, 2010; U.S. Provisional Patent Application No. 61/308,704 filed Feb. 26, 2010; U.S. Provisional Patent Application No. 61/332,413 filed May 7, 2010; U.S. Provisional Patent Application No. 61/380,003 filed Sep. 3, 2010; U.S. Provisional Patent Application No. 61/386,962 filed Sep. 27, 2010; U.S. Provisional Patent Application No. 61/407,454 filed Oct. 28, 2010; U.S. Provisional Patent Application No. 61/380,155 filed Sep. 3, 2010; and U.S. Provisional Patent Application No. 61/431,926 filed Jan. 12, 2011.

This application is a continuation-in-part of the following U.S. Provisional Application, which is incorporated herein by reference in its entirety: U.S. Provisional Patent Application No. 62/002,968 filed May 26, 2014.

### BACKGROUND OF THE INVENTION

Field of the Invention

Embodiments of the present invention generally relate to managing medical and non-medical treatments, and more particularly, a method and system for managing treatments comprising imaging organs, analyzing the captured images, profiling the human subjects based on the analyses, categorizing the human subjects based on the profiles, recommending treatments, therapies or regimens and health condition or care management procedures and tracking efficacy of the recommended treatments, therapies or regimens and health condition or care management procedures thereby facilitating customizing and optimizing treatments.

Description of the Related Art

Medical case management is a collaborative process that facilitates recommended treatment plans to assure the appropriate medical care is provided to disabled, ill or injured individuals. Medical case management refers to the planning and coordination of health care services appropriate to achieve the goal of medical rehabilitation. Medical case management may include, but is not limited to, care assessment, including personal interview with the injured individuals, and assistance in developing, implementing and coordinating a medical care plan with health care providers, as well as the individuals and his/her family and evaluation of treatment results. Medical case management requires the evaluation of a medical condition, development of a plan of care, implementation of the same, coordination of medical resources, communication of healthcare needs to an individual, monitoring the individual's progress and promotion of cost-effective care.

However, apparently one problem in connection with conventional medical case management may be the absence of best-in-class optimization via customization of recommended treatment plans based on efficacy tracking of the recommended treatment plans.

Therapy, often abbreviated tx or Tx, is the attempted remediation of a health problem, usually following a diagnosis. Therapies can be classified according to the method of treatment comprising at least one of (1) by usage of matter in turn comprising at least one of (1.1) by usage of drug that in turn comprises at least one of pharmacotherapy, chemotherapy, mesotherapy and a combination thereof; (1.2) by usage of medical device; (1.3) by usage of gene, namely gene therapy; (1.4) by usage of gold, namely chrysotherapy (aurotherapy); (1.5) by usage of hormone, namely hormone therapy; (1.6) by usage of organism, namely biotherapy, that in turn comprises at least one of (1.6.1) by usage of virus, namely virotherapy; (1.6.2) by usage of bacteriophage, namely phage therapy; (1.6.3) by usage of maggot, namely maggot therapy; (1.7) by usage of ozone, namely ozonotherapy; (1.8) by usage of salt, namely speleotherapy; (1.9) by usage of serum, namely serotherapy; (1.10) by usage of smell, namely aromatherapy; (1.11) by usage of water, namely hydrotherapy; and (1.12) by usage of plants, namely phytotherapy, and a combination thereof; (2) by usage of energy in turn comprising at least one of (2.1) by usage of electric energy that in turn comprises at least one of (2.1.1) by usage of electricity, namely electrotherapy, (2.1.2) by usage of electromagnetic radiation, namely electromagnetic therapy, (2.1.3) by usage of magnetic energy, namely magnet therapy, and a combination thereof; (2.2) by usage of light, namely phototherapy; (2.3) by usage of mechanical energy that in turn comprises at least one of manual therapy, namely massotherapy, therapy by exercise, namely physiotherapy, and a combination thereof; (2.4) by usage of sound that in turn comprises at least one of cymatic therapy, music therapy and a combination thereof; (2.5) by usage of radiation, namely radiotherapy; (2.6) by usage of temperature that in turn comprises at least one of by heat, namely thermotherapy, and by cold, namely by cryotherapy, and a combination thereof; (3) by human interaction in turn comprising at least one of (3.1) by counseling, namely psychotherapy; (3.2) by education that in turn comprises at least one of (3.2.1) by psychoeducation, (3.2.2) by usage of information therapy and a combination thereof; (3.3) by usage of physical therapy that in turn comprises at least one of occupational therapy, massage therapy, chiropractic, acupuncture and a combination thereof; (3.4) by lifestyle modifications that in turn comprises at least one of avoidance of unhealthy food, maintenance of a predictable sleep schedule and a combination thereof; (3.5) by coaching and a combination thereof; (4) by animal interaction that in turn comprises at least one of (4.1) by fish, namely ichthyotherapy, (4.2) by horse that in turn comprises at least one of equine therapy, hippotherapy and a combination thereof; (5) by meditation that in turn comprises at least one of meditative therapy, mindfulness and a combination thereof; (6) by reading, namely by bibliotherapy, and a combination of all the above. Further, therapies are classified in accordance with the method of treatment via implementation of one or more levels of care based on at least one of chronology, priority and intensity. Specifically, the levels of care classify healthcare into the categories of at least one of chronology, priority and intensity as follows: emergency care, intensive care, ambulatory care, home care, primary care, secondary care, tertiary care, end-of-life care including at least one of palliative and hospice care. Furthermore, therapies are classified in accordance with the method of treatment via implementation of one or more lines of therapy based on the priorities or rankings thereof, for instance at least one of a first-line therapy, second-line therapy, third-line therapy, and so on. Still further, therapies are classified in accordance with the method of treatment based on intent of treatment. Specifically, the therapies are classified in accordance with the method of treatment based on the intent of treatment as follows: abortive therapy, consolidation therapy, curative therapy, definitive therapy, empiric therapy, gold standard therapy, investigational therapy, maintenance therapy, palliative therapy, preventive therapy (prophylactic therapy), salvage therapy and supportive therapy, respectively. However, apparently one problem in connection with conventional therapies may be the absence of best-in-class optimization via customization of recommended therapies based on efficacy tracking of the recommended therapies.

Thus, there is a need for a method and system for managing treatments. Specifically, there is a need for a method and system for managing medical and non-medical treatments comprising imaging organs, analyzing the captured images, profiling the human subjects based on the analyses, categorizing the human subjects based on the profiles, recommending treatments, therapies or regimens and health condition or care management procedures and tracking efficacy of the recommended treatments, therapies or regimens and health condition or care management procedures thereby facilitating customizing and optimizing medical and non-medical treatments. More specifically, there is a need for the design and implementation of such system and the method therefor.

### SUMMARY OF THE INVENTION

Embodiments of the present invention generally relate to managing skin health, and more particularly, to imaging, analyzing, quantifying and classifying skin acne and recommending therapies and tracking efficacy of the recommended therapies.

These and other systems, processes, methods, objects, features, and advantages of the present invention will be apparent to those skilled in the art from the following detailed description of the preferred embodiment and the drawings. All documents mentioned herein are hereby incorporated in their entirety by reference.

Real-time analysis of digitally captured skin characteristics facilitates timely skin condition assessment, skin regimen recommendation, and skin regimen effectiveness tracking.

The problem of generating a skin condition assessment in real-time is solved by having a skin condition analysis module capable of doing real-time analysis of digital skin data, acquired partly using diffused reflectance spectroscopy and/or detecting the red-green-blue components of re-emitted white light.

In an aspect of the invention, a skin care device may include an electromagnetic radiation source capable of directing incident electromagnetic radiation to a location on the skin of a user, a radiation detector for measuring various parameters of radiation re-emitted from the location, and a skin condition analysis module coupled to the detector, the analysis module capable of generating a skin condition assessment in real-time, based partly on at least one of RGB analysis and diffused reflectance analysis of the radiation parameters. In the device, the incident electromagnetic radiation may include radiation in at least one of the visible, near-infrared, and near-ultraviolet spectrum. The incident radiation may include white light. In the device, the radiation parameters may include at least the degree of polarization of the re-emitted radiation. In the device, the radiation source may be a set of light emitting diodes. In the device, the skin condition assessment may also be partly based on analysis of a photographic image of a skin region surrounding the location. In the device, the device may be a miniature device. Miniature may mean that no dimension of the detector exceeds six inches. The device may further comprise a memory module for storing the skin condition assessment. The device may further comprise a user interface. The user interface may be operated using voice commands. In the device, skin assessment data of locations may be overlaid on an image of a larger skin region and displayed on the display surface. The device may further comprise an access restriction module used for restricting access to authorized users only. The access restriction module may be based on biometric access control. The device may be capable of generating alerts about abnormal skin conditions in real-time. The device may further comprise a skin care regimen recommendation module that generates a displayable skin care regimen recommendation. The skin care regimen recommendation may be based at least partly on determination of a skin profile of the user and use of skin care regimen recommendations of persons with a similar profile. The skin care regimen recommendation module may be linked to a product database. The product database may include products available in a point-of-sale location. The availability of a specific product recommended by the skin care regimen recommendation module may be indicated by an audio-visual signal. The device may further comprise a skin care regimen effectiveness module that generates a displayable skin care regimen effectiveness report. The device may further comprise a communication module for communicating with a remote computer. The communication may occur wirelessly. The communication may occur over an internet. The remote computer may be operable by a physician. The device may be wand-shaped. The device may be wearable by the user.

In an aspect of the invention, the skin care device may include an electromagnetic radiation source capable of directing incident electromagnetic radiation to a location on the skin of a user, a detector for measuring various parameters of radiation re-emitted from the location, a skin condition analysis module coupled to the detector, the analysis module capable of generating a skin condition assessment in real-time, based partly on at least one of RGB analysis and diffused reflectance analysis of the radiation parameters, and a display panel for reflecting the image of the user. In the device, the display panel may be touch-sensitive such that touching the location in a skin region image displayed in the display panel triggers display of a magnified image of the location. The device may further comprise a camera. The camera may be integral with the display panel. The camera may be wirelessly linked to the display panel. In the device, the display panel may be a mirror. In the device, a stored image of the user is used to automatically identify the person. The device may further comprise a user interface for controlling the skin care device. The user interface may be operated using voice commands. The device may further comprise a skin care regimen recommendation module capable of generating a displayable skin care regimen recommendation. The skin care regimen recommendation may be based at least partly on determination of a skin profile of the user and use of skin care regimen recommendations of persons with a similar profile. The device may further comprise a skin care regimen effectiveness module capable of generating a displayable skin care regimen effectiveness report.

In aspects of the invention, an imaging device permits a user to take high magnification pictures of the skin in the vicinity of an area of concern and submit those pictures, optionally along with textual and data responses, for medical, non-medical, and cosmetic analysis, diagnosis and treatment recommendation and follow-up.

In an aspect of the invention, a method and system of a non-invasive imaging device may comprise an illumination source comprising an incident light source to direct light upon skin; and a detector for detecting the degree of polarization of light reflected from the skin. In the method and system, the illumination source may be positioned to direct light at a selected angle alpha. Varying alpha may vary the depth of the measurement of the layers in the skin. Each depth may have a specific angle which produces a full polarized reflection. In the method and system, the incident light source may be an unpolarized light source. The unpolarized light may be white light, multiple selected wavelengths, or a single wavelength. The method and system may further comprise a sensor for capturing an image of the reflected or re-emitted light. The method and system may further comprise an optical facility for detecting reflected or re-emitted light from the skin. The method may determine both reflected or re-emitted light, and newly emitted light, through the process of absorption and re-emission. The method and system may further comprise a communication facility for transmitting the detected information. The method and system may further comprise a storage facility for storing information collected by the device.

In an aspect of the invention, a method and system for determining a skin state may comprise illuminating skin with an incident light source, detecting the degree of polarization of light reflected from the skin, and determining a skin state based on an aspect of the polarization of the reflected or re-emitted light. In the method and system, the incident light may be directed at a selected angle alpha. Varying alpha may vary the depth of the measurement of the layers in the skin. Each depth may have a specific angle which produces a full polarized reflection. In the method and system, the incident light source may be an unpolarized light source. The unpolarized light may be white light, multiple selected wavelengths, or a single wavelength. In the method of claim, the aspect of the polarization may be at least one of an orientation, an amplitude, a phase, an angle, a shape, a degree, an amount, and the like. In the method and system, determining may be done using an algorithm. The algorithm may involve artificial neural networks, non-linear regression, genetic algorithms, fuzzy logic, fractal and multi-fractal analysis, and the like. The methods and systems may further comprise filtering the reflected or re-emitted light to obtain polarized light of at least one wavelength defined by the filter output. The algorithmic analysis may be performed on the filtered image. In the method and system, determining may involve creating an image from the difference between the reflected diffusion light and the reflected polarized light. In the method and system, determining may involve comparing the aspect of the polarization of the reflected or re-emitted light to a calibration signal. In the method and system, determining may further comprise considering at least one of user input and a visual analysis.

In an aspect of the invention, a non-invasive imaging device may comprise an illumination source comprising an incident light source to direct light upon an area of concern and a detector for detecting the degree of polarization of light reflected from the area of concern. In the method and system, the illumination source may be positioned to direct light at a selected angle alpha. Varying alpha may vary the depth of the measurement of the layers in the skin. Each depth may have a specific angle which produces a full polarized reflection. In the method and system, the incident light source may be an unpolarized light source. The unpolarized light may be white light, multiple selected wavelengths, or a single wavelength. The method and system may further comprise a sensor for capturing an image of the reflected or re-emitted light. The method and system may further comprise an optical facility for detecting reflected or re-emitted light from the skin. The method and system may further comprise a communication facility for transmitting the detected information. The method and system may further comprise a storage facility for storing information collected by the device.

In an aspect of the invention, a method of determining moisture levels in the skin may comprise emitting incident light towards a skin structure, detecting a degree of polarization of the light induced by the skin structure, and determining a moisture level based on the amount of polarized and reflected or re-emitted light. The method and system may further comprise combining the assessment of moisture level with skin color measurements to determine luminosity. In the method and system, the incident light may be unpolarized light. The unpolarized light may be white light, light of multiple selected wavelengths, or of a single wavelength, or one or more monochromatic lights. In the method and system, determining may involve use of an algorithm. In the method and system, determining a moisture level may be based on the ratio of polarized and reflected or re-emitted light.

In an aspect of the invention, a method and system of determining elasticity of the skin may comprise emitting incident light towards a skin structure, detecting an aspect of polarization of the light reflected by the skin structure, correlating the aspect of polarization with a concentration of elastin, and determining elasticity level based on the elastin status. In the method and system, determining may involve use of an algorithm. In the method and system, the incident light may be unpolarized light. The unpolarized light may be white light, light of multiple selected wavelengths, or a single wavelength of light.

In an aspect of the invention, a method and system of determining firmness of the skin may comprise emitting incident light towards a skin structure, detecting an aspect of polarization of the light reflected by the skin structure, correlating the aspect of polarization with the status of at least one of an elastin, a collagen, and an activity of a sebaceous gland, and determining firmness based on the concentration of at least one of elastin and collagen and sebaceous gland activity. In the method and system, the sebaceous gland activity may be indicated by at least one of a number of glands, percent of glands open/closed, and level of clog/fill. In the method and system, correlating may involve use of an algorithm.

In an aspect of the invention, a method and system for obtaining dermal biophysical properties may comprise performing a spectral analysis of image data acquired from the degree of polarization of reflections and absorption and re-emission of incident light from skin structures, wherein the property is at least one of a structure, form, status, number, size, state, and stage of at least one of a: melanocyte, melanin, hemoglobin, porphyrin, triptofan, NADH, FAD, keratin, carotene, collagen, elastin, sebum, sebaceous gland activity, pore (sweat and sebaceous), moisture level, elasticity, luminosity, firmness, fine line, wrinkle count and stage, pore size, percent of open pores, skin elasticity, skin tension line, spot, skin color, psoriasis, allergy, red area, general skin disorder or infection, tumor, sunburn, rash, scratch, pimple, acne, strias, insect bite, itch, bleeding, injury, inflammation, photodamage, pigmentation, tone, tattoo, percent burn/burn classification, mole (naevi, nevus), aspect of a skin lesion (structure, color, dimensions/asymmetry), melanoma, automated follow-up of pigmented skin lesions, dermally observed disorder, cutaneous lesion, cellulite, boil, blistering disease, congenital dermal syndrome, (sub)-cutaneous mycoses, melasma, vascular condition, rosacea, spider vein, texture, skin ulcer, wound healing, post-operative tracking, melanocytic lesion, non-melanocytic lesion, basal cell carcinoma, seborrhoic keratosis, sebum (oiliness), nail- and/or hair-related concern, and the like.

In an aspect of the invention, a system and method may comprise providing an interface that includes a social networking domain or rating-and-ranking system and at least one of a skin state determination facility and a recommendation engine, and enabling users, either all or a selected few, of the interface to perform a skin state determination within the interface. In the method and system, the skin state determination facility may comprise capturing images with a non-invasive imaging device comprising an illumination source comprising an incident light source to direct light upon skin, and a detector for detecting the degree of polarization of light reflected from the skin, and determining a skin state based on an aspect of the polarization of the reflected or re-emitted light. The method and system may further comprise receiving product and regimen recommendations from the recommendation engine based on what other users with similar skin states are using as well as data regarding ingredients, effectiveness, safety, and the like. The method and system may further comprise comparing skin states, products, regimens, and recommended products or regimens with peers within the social networking domain of the interface. Comparing may comprise an analysis of similarity based on the spectral analysis of the degree of polarization of reflected or re-emitted light from users' skin. In the method and system, the interface may comprise a regimen tracker. The regimen tracker may be populated using a drag-and-drop or click-to-add functionality. In the method and system, the interface may comprise a rating facility or a product information facility. The product information facility may enable a user to obtain product information by search. Search may be a search of product identifiers, product ratings, drag-and-drop items, images, barcode scans, skin states, and profiles.

In an aspect of the invention, a method and system for determining a skin state may comprise obtaining the answers to a series of subjective questions regarding the skin, obtaining an objective skin analysis using a dermal imaging device, and combining the subjective and objective results algorithmically to obtain a skin state.

In an aspect of the invention, a system and method for providing recommendations for skin care based on a skin state and a skin care goal may comprise obtaining a skin state of an individual, categorizing the individual by skin state, and recommending products and regimens that are effective for other individuals of the category in achieving the skin care goal. In the method and system, the system may be operable over a network. In the method and system, the skin state may be determined based on analysis of the degree of polarization of light reflected from the skin of the individual.

In an aspect of the invention, a method for tracking the effectiveness of a skin care product or regimen may comprise obtaining a baseline skin state assessment, recommending a monitoring interval based on at least one of the skin care goal, product, and regimen, obtaining a second skin state assessment, comparing the second assessment to the baseline assessment to determine progress towards a skin care goal, and optionally, optimizing the regimen or product in order to improve a skin state. In the method and system, the skin assessment may be based on analysis of the degree of polarization of light reflected from the skin of the individual.

In an aspect of the invention, a personalized skin condition analysis system and related methods may comprise an imaging device, comprising an illumination source comprising an incident light source to direct light upon skin, and a detector for detecting the degree of polarization of light reflected from the skin, and a user interface for controlling the device. In the methods and system, the device may be adapted to interact with a physical interface to download image data to update a record of at least one of a practitioner, a spa, a salon, cosmetic sales, a cosmetics manufacturer, a clinical trials database, and a third party database. In the method and system, the illumination source may be positioned to direct light at a selected angle alpha. Varying alpha may vary the depth of the measurement of the layers in the skin. Each depth may have a specific angle which produces a full polarized reflection. In the method and system, the incident light source may be an unpolarized light source. The unpolarized light may be white light, multiple selected wavelengths, or a single wavelength. The method and system may further comprise a sensor for capturing an image of the reflected or re-emitted light. The method and system may further comprise an optical facility for detecting reflected or re-emitted light from the skin. The method and system may further comprise a communication facility for transmitting the detected information. The method and system may further comprise a storage facility for storing information collected by the device.

In an aspect of the invention, a non-invasive imaging device may comprise an illumination source comprising an incident light source to direct light upon skin; and a detector for detecting a characteristic of the light reflected from the skin. In the device, the illumination source may be positioned to direct light at a selected angle alpha. Varying alpha may vary the depth of the measurement of the layers in the skin. Each depth may have a specific angle which produces a full polarized reflection. In the device, the incident light source may be a polarized light source or unpolarized light source. The unpolarized light may be at least one of white light, light of a single wavelength, and light of multiple single wavelengths. The device may further comprise a sensor for capturing an image of the reflected or re-emitted light. The device may further comprise an optical facility for detecting reflected or re-emitted light from the skin. The device may further comprise a communication facility for transmitting the detected information. The device may further comprise a storage facility for storing information collected by the device. In the device, the reflected or re-emitted light may be at least one of polarized light and unpolarized light.

In an aspect of the invention, a method and system for determining a skin state may comprise illuminating skin with an incident light source; detecting a characteristic of the light reflected from the skin; and determining a skin state based on at least one characteristic of the reflected or re-emitted light. In the method and system, the incident light may be directed at a selected angle alpha. Varying alpha may vary the depth of the measurement of the layers in the skin. Each depth may have a specific angle which produces a full polarized reflection. In the method and system, the incident light may be unpolarized or polarized light. The unpolarized light may be at least one of white light, light of a single wavelength, and light of multiple single wavelengths. In the method and system, the reflected or re-emitted light may be at least one of polarized light and unpolarized light. In the method and system, the characteristic may be at least one of light source, light intensity, wavelength of light, angle of light, electrical and magnetic properties of the light, and polarization state of the light. An aspect of the polarization may be at least one of an orientation, an amplitude, a phase, an angle, a shape, a degree, and an amount. In the method and system, determining may be done using an algorithm. The algorithm may involve artificial neural networks, non-linear regression, genetic algorithms, fuzzy logic, or fractal and multi-fractal analysis. The method and system may further comprise filtering the reflected or re-emitted light to obtain light of a wavelength defined by the filter output. The analysis may be performed on the filtered image. In the method and system, determining may involve creating an image of the difference between reflected diffusion light and reflected polarized light. In the method and system, determining may involve comparing the aspect of the polarization of the reflected or re-emitted light to a calibration signal. In the method and system, determining may further comprise considering at least one of user input and a visual analysis.

In an aspect of the invention, a non-invasive imaging device may comprise an illumination source comprising an incident light source to direct light upon an area of concern; and a detector for detecting a characteristic of the light reflected from the area of concern. In the device, the illumination source may be positioned to direct light at a selected angle alpha. Varying alpha may vary the depth of the measurement of the layers in the skin. Each depth may have a specific angle which produces a full polarized reflection. In the device, the incident light source may be a polarized light source or unpolarized light source. The unpolarized light may be at least one of white light, light of a single wavelength, and light of multiple single wavelengths. The device may further comprise a sensor for capturing an image of the reflected or re-emitted light. The device may further comprise an optical facility for detecting reflected or re-emitted light from the skin. The device may further comprise a communication facility for transmitting the detected information. The device may further comprise a storage facility for storing information collected by the device. In the device, the reflected or re-emitted light may be at least one of polarized light and unpolarized light.

In an aspect the invention, a system and method may be used to determine healthy and melanocytic skin. The first, reflected spectrum and/or emission spectrum from sample which is skin malformation (SM), subtract reflected spectrum from normal healthy skin (SN). The second, from obtained resulting spectral plots (SM−SN) subtract reflected spectrum from adequate comparing screen, which represents spectral plot of the light source (SO). In that path appeared pure characteristics of change generated by skin. For differentiation between melanoma, other malignant or benign nevus and healthy skin can be used data on maxima, minima and zero positions, in wavelength scale and data on maxima and minima intensities.

In an aspect of the invention, a system and method may comprise capturing an image of a material illuminated with incident non-angled white light and angled white light, generating a normalized red and blue color channel histogram for each image, correlating the normalized red and blue color channel histograms to a wavelength scale to obtain red and blue color channel spectral plots, and convoluting the spectral plots by subtracting the spectral plot for angled light from the spectral plot for non-angled light for each color channel to generate red and blue normalized, composite color channel spectral plots, and subtracting the normalized, composite blue channel spectral plot from the normalized, composite red channel spectral plot to generate a spectral signature for the material. In the system and method, the illumination source may be positioned to direct light at a selected angle alpha. Varying alpha varies the depth of the measurement in the material. In the system and method, the unit scale on the spectral signature may be a difference of wavelength. In the system and method, the material is inorganic and/or organic matter. In the system and method, the spectral signature may be analyzed for at least one of number of peaks and troughs, amplitude and shape of peaks and intermediate structures and patterns. In the system and method, the spectral signature may be analyzed for metal composition, identification, purity, and strength. In the system and method, the spectral signature may be analyzed for water quality, composition, and purity. In the system and method, elements of the spectral signature may be tagged and tracked over time in order to track changes in the characteristics of the material. In the system and method, the spectral signature may be analyzed to measure, track or monitor a skin state. In the system and method, the spectral signature may be useful for the counterfeit analysis of money. In the system and method, the spectral signature may be analyzed for at least one of sweat gland activity and anti-perspirant effectiveness. In the system and method, the spectral signature may be analyzed for Mad Cow disease. In the system, the spectral signature may be analyzed for food, all epidermal diseases, melanoma and skin cancers, rheumatoid diseases, and all diseases that show on the skin. In the system and method, the spectral signature may be useful for monitoring post-operative cosmetic concerns. In the system and method, the spectral signature may be useful for predicting and monitoring secretion from the mammary glands of lactating women. In the system and method, the spectral signature may be fed into a recommendation engine to provide feedback and modifications to aspects of a regimen. In the system and method, the wavelength position of ideal blue in Maxwell's color triangle is aligned with the wavelength position of ideal red in Maxwell's color triangle when convoluting the composite spectral plots to obtain the spectral signature.

A method and a system are disclosed for determining skin characteristics and cosmetic features. A minimal error output is generated. In accordance with exemplary embodiments of the present invention, according to a first aspect of the present invention, a method for determining skin characteristics and cosmetic features using color analysis may include a step of analyzing color of skin images in a pixel by pixel manner in a Red Green Blue (RGB) color system for an acquired digital image. The step of analyzing color of skin images in a pixel by pixel manner in a RGB color system for an acquired digital image may include analyzing a picture of a part of a person's skin by generating a table of most frequent colors appearing in the picture.

According to the first aspect, a method for determining skin characteristics and cosmetic features using color analysis includes a step of generating a sample of most frequent standard RGB (sRGB) colors responsive to analyzing color of skin images in a pixel by pixel manner in the RGB color system for the acquired digital image after converting colors obtained in device dependent RGB color system into device independent standard RGB color system (sRGB). The step of generating a sample of most frequent sRGB colors responsive to analyzing color of skin images in the sRGB color system for the acquired digital image may include preserving a plurality of sRGB color values.

In this embodiment of the invention, the sRGB color system may be used for image analysis. Determination of other skin characteristics, melanoma, skin related tumors and skin related disorders require image analysis based on other color systems such as YIQ, YCbCr, L*a*b*, L*u*v* and HSL/HSV. The enhancement of the current algorithm may include at least one of these color systems and its/their correlation with presented sRGB analysis.

According to the first aspect, a method for determining skin characteristics and cosmetic features using color analysis includes a step of modeling the R, G and B component color distribution with Gaussian probabilistic distribution with estimated parameters (expected value and standard deviation) on the generated sRGB color sample for the acquired digital image further including approximating colors on the generated sRGB color samples by a Gaussian normal distribution. In accordance with an exemplary embodiment of the present invention the step of approximating colors on the generated sRGB color samples by a Gaussian normal distribution comprises approximating colors on the generated sRGB color samples by a superposition of a plurality of Gaussian normal distributions.

According to the first aspect, a method for determining skin characteristics and cosmetic features using color analysis includes a step of generating a phototype of the skin through a decision tree unit responsive to the estimated distribution model parameters colors. The phototype of the skin may be generated according to a corrected Fitzpatrick classification. In accordance with an exemplary embodiment of the present invention, the step of generating phototype of the skin according to corrected Fitzpatrick classification includes generating phototype of the skin according to a skin type scale which ranges from very fair skin to very dark skin. This method may be measured both on the most exposed region and relate to the current level of phototype based on level of tan on the skin.

According to a second aspect, a system for skin phototype determination using photograph analysis may be disclosed. The system may include an image capturing device for capturing digital images of a skin. The image capturing device may include a digital camera unit.

According to the second aspect, the system for skin phototype determination using photograph analysis may include an analyzer coupled to the image capturing device for performing a pixel by pixel analysis of a picture of a part of a person's skin. The analyzer may include a quantization device for generating a look-up table of most frequent colors appearing on the picture of the part of the person's skin.

According to the second aspect, the system for skin phototype determination using photograph analysis may include a sampling device coupled to the image capturing device for generating standard Red Green Blue (sRGB) color samples for the captured digital image of the skin.

According to the second aspect, the system for skin phototype determination using photograph analysis may include an approximating device coupled to the sampling device for approximating the color distribution parameters on the generated sRGB color samples using the estimates of expected value and standard deviation for the captured digital image of the skin. The approximating device may include at least one Gaussian normal distribution unit.

According to the second aspect, the system for skin phototype determination using photograph analysis may include a decision tree unit coupled to the approximating device for generating a phototype of the skin using Red and Blue components of the approximated colors. The decision tree unit may include a Fitzpatrick scaling unit for categorizing a skin phototype in accordance with a skin type scale which ranges from very fair skin to very dark skin.

According to the second aspect, an exemplary embodiment of the present invention discloses a scaled Gaussian normal distribution unit for approximating colors on the generated sRGB color samples using estimates of expected value and standard deviation for the captured digital image of the skin.

According to the second aspect of the present invention, the system for skin phototype determination using photograph analysis may include a subsystem for determination of cosmetic features for a human element and a veterinary element. The cosmetic features may further include features pertaining to hair, nail and skin.

In another aspect the system may include a sampling device for generating standard Red Green Blue color samples of the captured digital image of the skin, the generated samples of standard Red Green Blue are in the range of values between 0 and 255 and they are preserved for further processing.

In another aspect the system may include an approximating device coupled to the sampling device for approximating the color distribution parameters on the generated sRGB color samples in the range of values between 0 and 255 by Gaussian normal distribution using the estimates of expected value and standard deviation for the captured digital image of the skin.

In another aspect the system may further include a decision tree unit coupled to the approximating device for generating a phototype of the skin using standard Red and Blue components of the approximated colors, the decision tree unit with an algorithm equates estimates of expected values and standard deviation for the captured image of the skin to the Fitzpatrick notation of skin analysis for determination of skin phototype.

In another aspect the system may automatically adjust lighting intensity and wavelengths and angles in order to assess various factors of the skin.

In yet another aspect of the system skin phototype may be determined using photograph analysis for use in cosmetics and surgical industry.

In an aspect of the invention, a skin care device may include an electromagnetic radiation source capable of directing incident electromagnetic radiation to a location on the skin of a user, a radiation detector for measuring various parameters of radiation re-emitted from the location, and a skin condition analysis module coupled to the detector, the analysis module capable of generating a skin condition assessment in real-time, based partly on at least one of RGB analysis and diffused reflectance analysis of the radiation parameters. In the device, incident electromagnetic radiation may include radiation in at least one of the visible, near-infrared, and near-ultraviolet spectrum. The incident radiation may be white light. In the device, the radiation parameters include at least the degree of polarization of the re-emitted radiation. In the device, the radiation source may be a set of light emitting diodes. In the device, the skin condition assessment may be also partly based on analysis of a photographic image of a skin region surrounding the location. In the device, the device may be a miniature device. Miniature may mean that no dimension of the detector exceeds six inches. The device may further include a memory module for storing the skin condition assessment. The device may further include a user interface. The device may further include a display surface. The skin assessment data of locations may be overlaid on an image of a larger skin region and displayed on the display surface. The device may further include an access restriction module used for restricting access to authorized users only. The access restriction module may be based on biometric access control. The device may be capable of generating alerts about abnormal skin conditions in real-time. The user interface may be operated using voice and/or eye movement commands. The device may further include a skin care regimen recommendation module that generates a displayable skin care regimen recommendation. The skin care regimen recommendation may be based at least partly on determination of a skin profile of the user and use of skin care regimen recommendations of persons with a similar profile. The skin care regimen recommendation module may be linked to a product database. The product database may include products available in a point-of-sale location. The availability of a specific product recommended by the skin care regimen recommendation module may be indicated by an audio-visual signal. The device may further include a skin care regimen effectiveness module that generates a displayable skin care regimen effectiveness report. The device may further include a communication module for communicating with a remote computer. The communication may occur wirelessly. The communication may occur over an internet. The remote computer may be operable by a physician. The device may be wand-shaped. The device may be wearable by the user.

In an aspect of the invention, the device an electromagnetic radiation source capable of directing incident electromagnetic radiation to a location on the skin of a user, a detector for measuring various parameters of radiation re-emitted from the location, a skin condition analysis module coupled to the detector, the analysis module capable of generating a skin condition assessment in real-time, based partly on at least one of RGB analysis and diffused reflectance analysis of the radiation parameters, and a display panel for reflecting the image of the user. In the device, the display panel may be touch-sensitive such that touching the location in a skin region image displayed in the display panel triggers display of a magnified image of the location. The skin care device may further include a camera. The camera may be integral with the display panel. The camera may be wirelessly linked to the display panel. In the device, the display panel may be a mirror. In the device, a stored image of the user may be used to automatically identify the person. The device may further include a user interface for controlling the skin care device. The user interface may be operated using voice and/or eye movement commands. The device may further include a skin care regimen recommendation module capable of generating a displayable skin care regimen recommendation. The skin care regimen recommendation may be based at least partly on determination of a skin profile of the user and use of skin care regimen recommendations of persons with a similar profile. The device may further include a skin care regimen effectiveness module capable of generating a displayable skin care regimen effectiveness report.

In an aspect of the invention, a system and method for moving information objects available on a website to a receptacle to communicate with a plurality of people in a controlled access community network may include enabling movement of a plurality of information objects from a predetermined website to a web based network responsive to a regimen of a person, a routine of a person, a purpose of use of an information object of the plurality of information objects and a degree of affinity of a first person towards a second person, initiating at least one customized action from the actions including a drop down movement; a drag and drop movement for populating data; and a pop-up movement in a Graphical User Interface (GUI) responsive to enabling movement of a plurality of information objects from a predetermined healthcare website, and enabling transportation of the plurality of information objects across a plurality of websites. In the system and method, the plurality of information objects may pertain to a questionnaire on at least one of a human skin condition, product information, an article, a blog posting, an image, a video, an individual message, a forum posting, and a veterinary skin condition. In the system and method, the plurality of information objects pertains to a questionnaire on human cosmetic parameters and veterinary cosmetic parameters. The questionnaire on human cosmetic parameters and veterinary cosmetic parameters may include questions on at least one of a human nail and a veterinary nail. The questionnaire on human cosmetic parameters and veterinary cosmetic parameters may include questions on at least one of a human hair and a veterinary hair. In the system and method, the purpose of use of the information object may pertain to controlling at least one of cleansing, protection, repair, moisturizing, elasticity, firmness, glow, luminosity, anti-inflammatory properties, anti-itch properties, anti-wrinkle properties, firming, exfoliating, anti-redness properties, oil controlling, anti-aging properties and shine of a human skin. In the system and method, the degree of affinity of a first person towards a second person comprises at least one of a relationship of friendship between the first person and the second person; a genetic similarity between the first person and the second person; a similarity of lifestyle between the first person and the second person; a climatic similarity between a first residential environment and a second residential environment; and a skin type similarity between the first person and the second person. In the system and method, the step of enabling transportation of the plurality of information objects across a plurality of websites may include a sub-step of dragging an item of user interest off a website of the plurality of websites in a predetermined format and transferring through an electronic signal to affiliates of a user accessing the website. The affiliates of the user may be friends and relatives of the user or associated experts. In the system and method, the step of enabling movement of a plurality of information objects from a predetermined website to a web based network may include a sub-step of enabling drop down menus on the Graphical User Interface (GUI) responsive to a plurality of end user convenience and requirement parameters. In the system and device, the plurality of people in a web based network includes a plurality of people in an online friendship network. In the system and device, the plurality of people in a web based network includes a plurality of people in an online social network.

In an aspect of the invention, an interface including a social networking domain and at least one skin health assessment and recommendation unit for enabling users of the interface to perform a skin health assessment within the interface and to receive product and regimen recommendations from a recommendation engine based on a predetermined usage of health assessment and maintenance data may include a regimen tracker populated using a drag and drop facility, a rating unit for rating a plurality of healthcare facilities, and a product information unit for enabling a user to obtain product information by conducting a web based search of a plurality of web based drag and drop products, web based images and bar code scans. In the interface, the regimen tracker includes a diet tracking unit. In the interface, the plurality of healthcare facilities comprises at least one of skin cleansing, skin protection, skin moisture control, skin repair, skin elasticity, skin luminosity, skin firmness, skin wrinkles, pore size on skin, spots on skin, glow on skin, hair color, hair type, age and life stage further including marriage, pregnancy, dating and social life. In the interface, the product information comprises at least one of a product type, a product function, a product format, a product appropriateness level, a regimen information, product articles, product blogs, product safety, product toxicity, a product effectiveness index, a product cost information, and a product timeliness information. In the interface, the interface is a multiple language and customized interface for: web based applications; mobile phone applications; touch screen applications; and personal digital assistant applications. In the interface, the interface is seamlessly coupled with a dermal imaging device for customized web based access, control and maintenance of spectral analysis of image data acquired from a degree of polarization of reflections and re-emission of incident light from skin structures. The degree of polarization of reflections and/or re-emissions of incident light from skin structures is derived from at least one of a Red Green Blue (RGB) color analysis of a plurality of digital images; and an analysis from spectroscopic data image analysis.

In an aspect of the invention, a system and method for determining a health state may include obtaining the answers to a series of subjective questions regarding health conditions, obtaining an objective health assessment report through a dermal imaging device, and generating a combination of answers to the series of subjective questions and the objective health assessment report to thereby generate a health state output and a real skin type output. In the system and method, a real skin type output is generated based on biophysical properties generated by at least one of a person seeking skin health monitoring, a spa, and a cosmetic advisor. In the system and method, the objective health assessment report may include an objective skin health assessment report on at least one of systemic hydration, skin hydration, skin firmness, skin wrinkles, pore size on skin, spots on skin, glow on skin, melanocyte, melanin, hemoglobin, porphyrin, triptofan, NADH, FAH, keratin, carotene, collagen, elastin, sebum, sebaceous gland activity, sweat pore, sebaceous pore, moisture level, elasticity, luminosity, firmness, fine line, wrinkle count, pore size, percent of open pores, skin elasticity, skin tension line, spots, viscosity, epidermal, dermal sebum levels, skin color, psoriasis, allergy, red area, general skin disorder, infection, tumor, sunburn, rash, scratch, pimple, acne, insect bite, itch, bleeding, injury, inflammation, photodamage, pigmentation, tone, tattoo, percent burn, burn classification, mole, aspect of a skin lesion, melanoma, dermally observed disorder, cutaneous lesion, cellulite, strias, current tan level, boil, blistering disease, congenital dermal syndrome, cutaneous mycoses, melasma, vascular condition, rosacea, spider vein, texture, skin ulcer, wound healing, post-operative tracking, melanocytic lesion, nonmelanocytic lesion, basal cell carcinoma, seborrhoic keratosis, sebum hair color, hair type, nail condition, and age and life stage further including marriage, pregnancy, dating and social life. In the system and method, the objective health assessment report is sent to an end user through at least one of email, SMS, MMS, mobile phone, a graphical user interface (GUI) of an internet connected device, and a touch screen enabled personal digital assistant. The system and method may further include obtaining health assessment and maintenance data from a physiologically polarized light data. The step of obtaining health assessment and maintenance data from a physiologically polarized light data comprises obtaining health assessment and maintenance data from a Red Green Blue (RGB) color analysis device, wherein the data comprise at least one of a white light data, a blue light data, and an ultra violet light data. The step may further comprise obtaining at least one of the white light data, the blue light data, and the ultra violet light data by reading and recording conditions of at least one of the dermis and epidermis. Obtaining health assessment and maintenance data from a physiologically polarized light data comprises obtaining data pertaining to age, geography and demography for a person subjected to health monitoring.

In an aspect of the invention, a web-enabled health tracking method and system may include a camera comprising a photo guide unit for generating notes for each photograph captured, an interface coupled between the camera and a web-enabled computing system for uploading the photograph captured by the camera, a graphical user interface unit included in the web-enabled computing system for generating a frequently asked questionnaire unit further comprising a self answer guide module, a scoring module coupled to the frequently asked questionnaire unit, a comparison module coupled to the scoring module for comparing: a color parameter; a symmetry parameter; and a border parameter, an automation unit coupled to the graphical user interface for enabling a time-based synchronization of the frequently asked questionnaire unit, the scoring module, and the comparison module, and a learning unit coupled to the automation unit for activating: a user training module, an article module coupled to the user training module, a blogging unit coupled to the user training module and the article module, and a report unit including an email unit for emailing health related information. In the system and method, the camera comprises a tracking unit for tracking at least one of skin spots over time, laser treatment effectiveness, cellulite content in skin, current tan level, condition of veins and capillaries, botox treatment effectiveness, anti-aging treatment effectiveness, anti-acne treatment effectiveness, and a pictorial history of skin to be given to the doctor. The skin spots over time include at least one of blemishes, scars, rashes, lesions, and moles. In the system and method, the web-enabled computing system for uploading the photograph captured by the camera further includes a walkthrough module for walking through features of a skin health record of a first time user of the system, a personal skin photo album for reviewing pictorial history of a regular user of the system, and a product quality menu for tracking product expiration dates. In the system and method, the interface for uploading the photograph further includes a reminder unit for next photo for a regular user of the system; and a cosmetic status unit coupled to the reminder unit for displaying a current usage of a cosmetic for the regular user of the system. The current usage comprises a usage of at least one of a moisturizer, an antiseptic, a toner, a laser, and a botox. The system and method may further include a photo review unit for date based reviewing of at least one of a condition of a predetermined body part, a current usage status of a cosmetic, and a recommended usage list of cosmetics. In the system and method, the report unit may further include a secure transmission unit for sending a health assessment report to a medical practitioner, an affinity unit for discussing health assessment data with a friend, and a printing unit for printing health assessment data.

In an aspect of the invention, a mobile device-based health assessment system and method may include a photograph capturing device for capturing a skin image of a mobile device user, a transmission unit coupled with the photograph capturing device for uploading the captured skin image to a network location, a global positioning device coupled to the photograph capturing device for determining a location of the photograph capturing device, and a weather estimation device coupled to the photograph capturing device to determine a weather condition at a location of the mobile device user to thereby obtain a remote diagnosis report. In the system and method, the photograph capturing device further comprises at least one of a skin photograph assessment unit, a nail photograph assessment unit, and a hair photograph assessment unit. In the system and method, the global positioning device comprises a location tracker for answering user raised questions pertaining to geographical positioning of the user. In the system and method, the location tracker includes a database pertaining to weather intensive cosmetics. The system and method may further include a phone number tracker for enabling a mobile device user to contact health assessment and cosmetic outlets.

In an aspect of the invention, a system and method for estimation of skin type and skin features to create a unique spectral signature may include convoluting data from a first image captured in incident diffuse white light, wherein the data relate to reflected and/or re-emitted polarized or white light, convoluting data from a second image captured in incident polarized light, wherein the data relate to reflected and/or re-emitted polarized light, comparing extreme positions of at least two unique convolutions generated by convoluting data from the first image and the second image, and determining a distance between minimum and maximum intensity positions in convoluted red minus blue spectral plots from the at least two unique convolutions for generating a numerical skin type output. In the system and method, the physiological white light comprises three spectral intervals including a width less than 100 nanometer. The three spectral intervals pertain to red, green, and blue (RGB) colors. The three spectral intervals provide a natural white light sensation to a human eye. In the system and method, the step of comparing extreme positions of at least two unique convolutions comprises comparing a component (R−B)(W−P) for the reflected and/or re-emitted polarized light, and a component (R−B)W for the white light. The two unique convolutions in white light and polarized light further include a White Red component (WR), a White Blue component (WB), a reflected and/or re-emitted Polarized Blue component (PB) and a reflected and/or re-emitted Polarized Red component (PR). The two unique convolutions are based on a numerical value difference correlating to medical standards. The system and method may further include a spectral convolution scheme wherein multiple combinations of subtraction of blue spectrum from red, in white light and polarized white light are determined, wherein the spectral interval is expressed in a wavelength scale interval of 100 nanometers to 300 nanometers.

In an aspect of the invention, a system and method for creating a unique spectral signature of skin features may include a RGB (Red Green Blue) color channel spectral plot generated from digital images including single wavelength light matter interaction thereby generating skin type characterization output, skin moisture conductivity and skin elasticity in numerical and descriptive standards. In the system and method, the RGB (Red Green Blue) color channel spectral plots generated from digital images include multi-wavelength light matter interaction.

In an aspect of the invention, a system and method to track and store movement parameters of an imaging device moving over a subject area may include the steps of capturing an image of the subject area at a plurality of locations, identifying a direction of movement of the imaging device using an image processing technique for at least one captured frame, recognizing the direction of movement of the imaging device by comparing each frame with at least three distinct features captured to thereby triangulate a location of the imaging device, and comparing data of the captured image with a predetermined image database to store the image of the subject area and to store placement parameters of the imaging device. In the system and method, the step of capturing the image of the subject area at a plurality of locations comprises a sub step of capturing a continuous video image of the subject area. In the system and method, the step of capturing the image of the subject area at a plurality of locations comprises a sub step of capturing a frame by frame sequence of images of the subject area. In the system and method, the step of identifying a direction of movement of the imaging device using an image processing technique comprises a sub-step of a frame by frame comparison of the captured image to identify movement parameters of the imaging device. In the system and method, the step of recognizing the direction of movement of the imaging device by comparing each frame with at least three distinct features captured to triangulate a location of the imaging device comprises a sub-step of capturing a direction of movement of the imaging device by comparing three or more distinct positions across different frames.

In an aspect of the invention, an automated location tracking and data storage method and system for an imaging device may include an image capturing unit, a positioning unit coupled to the image capturing unit for positioning the imaging device on a subject area, and an image processing unit for enabling a frame by frame comparison of the captured image and for enabling the imaging device to capture three or more distinct points to triangulate a location of the imaging device to identify a direction of movement of the imaging device. In the system and method, the image capturing unit comprises a digital camera. In the system and method, the image capturing unit comprises at least one of a mobile device and a Personal Digital Assistant (PDA). In the system and method, the image processing unit comprises a comparison unit for comparing positions of three or more distinct points across different frames to capture direction of movement of the imaging device. The system and method may further include a sub-system for measuring lateral motion of the image capturing unit from a predetermined point to a new location on the subject area.

In an aspect of the invention, a system and method for determining a surgical excision margin may include illuminating a melanocytic lesion skin with an incident light source, detecting a characteristic of the light reflected and/or re-emitted from the melanocytic lesion, and determining a border between the melanocytic lesion and surrounding healthy tissue based on at least one characteristic of the reflected and/or re-emitted light. In the system and method, the incident light is directed at a selected angle alpha. In the system and method, varying alpha varies the depth of the measurement of the layers in the melanocytic lesion. Each depth has a specific angle which produces a full polarized reflection. In the system and method, the incident light is unpolarized light. The unpolarized light is at least one of white light, light of a single wavelength, and light of multiple single wavelengths. In the system and method, the incident light is polarized light. In the system and method, the reflected and/or re-emitted light is at least one of polarized light and unpolarized light. In the system and method, the characteristic is at least one of light source, light intensity, wavelength of light, angle of light, electrical and magnetic properties of the light, and polarization state of the light. An aspect of the polarization is at least one of an orientation, an amplitude, a phase, an angle, a shape, a degree, and an amount. In the system and method, determining is done using an algorithm. The algorithm involves at least one of artificial neural networks, fuzzy logic, fractal and multi-fractal analysis, non-linear regression, a genetic algorithm, white light analysis and RGB color analysis. The system and method may further include filtering the reflected and/or re-emitted light to obtain light of a wavelength defined by the filter output. Algorithmic analysis is performed on the filtered image. In the system and method, determining involves creating an image of the difference between reflected diffusion light and reflected polarized light. In the system and method, determining involves comparing the aspect of the polarization of the reflected and/or re-emitted light to a calibration signal. In the system and method, determining further comprises considering at least one of user input and a visual analysis.

Embodiments of the present invention disclose a method of managing skin health comprising capturing images of the skin, analyzing the captured images, determining at least one of susceptibility to acne and presence of acne, upon determination of the presence of acne, measuring the level of severity of acne, categorizing acne based on the measured level of severity, recommending at least one of skincare products, solutions and therapeutic regimens based on the measured level of severity of the acne, and tracking efficacy of the recommended the at least one of skincare products, solutions and therapeutic regimens.

Embodiments of the present invention disclose a method of managing acne comprising determining at least one of susceptibility to acne and presence of acne, upon determining susceptibility to acne, guesstimating the probability of occurrence of acne, upon determining presence of acne, estimating the current state of acne and guesstimating the expected state of acne, profiling the overall state of acne based on the current and expected state of acne, providing evidence-based recommendations for at least one of skincare products, solutions and therapeutic regimens based on the overall state of acne and tracking efficacy of the at least one of recommended skincare products, solutions and therapeutic regimens.

Embodiments of the present invention disclose a method of managing risk number for acne comprising determining presence of acne, upon determining presence of acne, estimating a current state of acne and guesstimating an expected state of acne, generating notifications based on an overall state of acne, determining factors controlling the risk number, determining regimens for providing recommendations for controlling the risk number based on the overall state of acne and determined factors controlling the risk number, implementing the recommended regimens, providing at least one of visual and quantitative indicators for at least one of increase and decrease in the risk number upon implementation of the recommended regimens and tracking efficacy of the recommended regimens based on the quantitative indicators for at least one of increase and decrease in the risk number.

Embodiments of the present invention a method for determining overall skin health comprising capturing one or more images of the skin illuminated with non-polarized (W) and reflected polarized (P) optical electromagnetic signals, performing spectral analysis of the one or more images acquired from the degree of polarization of at least one of reflection, absorption and re-emission of the non-polarized (W) and reflected polarized (P) optical electromagnetic signals from the skin to obtain pluralities of biophysical properties of the skin, wherein the biophysical properties are at least one of a structure, form and status of skin, and stage of at least one of: melanocyte, melanin, hemoglobin, porphyrins, tryptophan, nicotinamide adenine dinucleotide (NADH), flavin adenine dinucleotide (FAD), keratin, carotene, collagen, elastin, sebum, sebaceous gland activity, pore, sweat, sebaceous, moisture level, elasticity, luminosity, firmness, fine line, wrinkle count and stage, pore size, percent of open pores, skin elasticity, skin tension line, spot, skin color, psoriasis, allergy, red area, general skin disorder or infection, tumor, sunburn, rash, scratch, pimple, acne, stria, insect bite, itch, bleeding, injury, inflammation, photodamage, pigmentation, tone, tattoo, at least one of percent burn and burn classification, mole, nevus, aspects of a skin lesion, such as structure, color, dimensions and asymmetry, melanoma, automated follow-up of pigmented skin lesions, dermally observed disorder, cutaneous lesion, cellulite, boil, blistering disease, congenital dermal syndrome, (sub)-cutaneous mycoses, melasma, vascular condition, rosacea, spider vein, texture, skin ulcer, wound healing, post-operative tracking, melanocytic lesion, non-melanocytic lesion, basal cell carcinoma, seborrhoic keratosis, sebum (oiliness), nail and hair-related concern, and the like, generating a normalized Red (R) and Blue (B) color channel histogram for each of the one or more images, correlating the normalized Red (R) and Blue (B) color channel histograms to a wavelength scale to obtain Red (R) and Blue (B) color channel spectral plots and convoluting the spectral plots by subtracting the spectral plot for the polarized optical electromagnetic signal from the non-polarized optical electromagnetic signal for each color to generate Red (R) and Blue (B) normalized, composite color channel spectral plots and subtracting the normalized, composite Blue (B) channel spectral plot from the normalized, composite Red (R) channel spectral plot thereby resulting in generation of a spectral signature for the skin.

Embodiments of the present invention disclose a method for diagnosing acne comprising capturing one or more images of the skin illuminated with non-polarized (W) and reflected polarized (P) optical electromagnetic signals, performing spectral analysis of the one or more images acquired from the degree of polarization of at least one of reflection, absorption and re-emission of the non-polarized (W) and reflected polarized (P) optical electromagnetic signals from the skin to detect presence of at least one of acne-philic and acne-phobic genetic variants of Propionibacterium acnes based on strain-level analysis, generating a normalized Red (R) and Blue (B) color channel histogram for each of the one or more images, correlating the normalized Red (R) and Blue (B) color channel histograms to a wavelength scale to obtain Red (R) and Blue (B) color channel spectral plots and convoluting the spectral plots by subtracting the spectral plot for the polarized optical electromagnetic signal from the non-polarized optical electromagnetic signal for each color to generate Red (R) and Blue (B) normalized, composite color channel spectral plots and subtracting the normalized, composite Blue (B) channel spectral plot from the normalized, composite Red (R) channel spectral plot thereby resulting in generation of a spectral signature for the at least one of acne-philic and acne-phobic genetic variants of Propionibacterium acnes.

Embodiments of the present invention disclose a non-invasive method for diagnosing acne comprising generating optical electromagnetic signals, selectively filtering one or more wavelengths of the optical electromagnetic signals for illuminating skin, selectively filtering one or more wavelengths of the optical electromagnetic signals re-emitted from the skin owing to fluorescence, detecting a first set of wavelengths in a first range and a second set of wavelengths in a second range thereby facilitating detection of bacterial and non-bacterial acne and distinction therebetween, upon detecting presence of acne, determining level of severity of acne by performing at least one of acne segmentation based on establishment of evidence-based acne grading criteria, profiling the overall state of acne based on the level of severity of acne, providing evidence-based recommendations for at least one of skincare products, solutions and therapeutic regimens based on the level of severity of acne and tracking efficacy of the at least one of recommended skincare products, solutions and therapeutic regimens.

Embodiments of the present invention disclose a method for diagnosing acne comprising capturing one or more images of the skin illuminated with non-polarized (W) and reflected polarized (P) optical electromagnetic signals, performing spectral analysis of the one or more images acquired from the degree of polarization of at least one of reflection, absorption and re-emission of the non-polarized (W) and reflected polarized (P) optical electromagnetic signals from the skin to detect presence of at least one of acne-philic and acne-phobic genetic variants of Propionibacterium acnes based on strain-level analysis, generating a normalized Red (R) and Blue (B) color channel histogram for each of the one or more images, correlating the normalized Red (R) and Blue (B) color channel histograms to a wavelength scale to obtain Red (R) and Blue (B) color channel spectral plots and convoluting the spectral plots by subtracting the spectral plot for the polarized optical electromagnetic signal from the non-polarized optical electromagnetic signal for each color to generate Red (R) and Blue (B) normalized, composite color channel spectral plots and subtracting the normalized, composite Blue (B) channel spectral plot from the normalized, composite Red (R) channel spectral plot thereby resulting in generation of a spectral signature for the at least one of acne-philic and acne-phobic genetic variants of Propionibacterium acnes.

Embodiments of the present invention disclose a method for early prognosis of acne comprising detecting presence of at least one of acne-philic and acne-phobic genetic variants of Propionibacterium acnes using spectral signatures of the acne-philic and acne-phobic genetic variants of Propionibacterium acnes, monitoring the population of acne-phobic genetic variants of Propionibacterium acnes over a period of time to determine at least one of increase and decrease in the population thereof, upon determining at least one increase in the population of acne-philic of genetic variants of Propionibacterium acnes and decrease in the population of acne-phobic genetic variants of Propionibacterium acnes over the period of time and forecasting propensity to acne at an early stage based on at least one of the rate of increase in the population of acne-philic of genetic variants of Propionibacterium acnes and decrease in the population of acne-phobic genetic variants of Propionibacterium acnes over the period of time.

Embodiments of the present invention disclose a method for determining likelihood of treatment of acne comprising capturing one or more images of the skin illuminated with non-polarized (W) and reflected polarized (P) optical electromagnetic signals, performing spectral analysis of the one or more images acquired from the degree of polarization of at least one of reflection, absorption and re-emission of the non-polarized (W) and reflected polarized (P) optical electromagnetic signals from the skin to detect presence of at least one of families of bacteriophage, generating a normalized Red (R) and Blue (B) color channel histogram for each of the one or more images, correlating the normalized Red (R) and Blue (B) color channel histograms to a wavelength scale to obtain Red (R) and Blue (B) color channel spectral plots and convoluting the spectral plots by subtracting the spectral plot for the polarized optical electromagnetic signal from the non-polarized optical electromagnetic signal for each color to generate Red (R) and Blue (B) normalized, composite color channel spectral plots and subtracting the normalized, composite Blue (B) channel spectral plot from the normalized, composite Red (R) channel spectral plot thereby resulting in generation of a spectral signature for the at least one of families of bacteriophage.

Embodiments of the present invention disclose a method for fluorescence-based imaging of a target comprising illuminating the target with a light source emitting light of at least one wavelength or wavelength band causing at least one biomarker to fluoresce, detecting fluorescence of the at least one biomarker with an image detector, generating optical electromagnetic signals, performing spectral analysis of the one or more images acquired from the degree of polarization of at least one of reflection, absorption and re-emission of the non-polarized (W) and reflected polarized (P) optical electromagnetic signals from the skin to obtain pluralities of biophysical properties of the skin, wherein the biophysical properties are at least one of a structure, form and status of skin, and stage of at least one of: melanocyte, melanin, hemoglobin, porphyrins, tryptophan, nicotinamide adenine dinucleotide (NADH), flavin adenine dinucleotide (FAD), keratin, carotene, collagen, elastin, sebum, sebaceous gland activity, pore, sweat, sebaceous, moisture level, elasticity, luminosity, firmness, fine line, wrinkle count and stage, pore size, percent of open pores, skin elasticity, skin tension line, spot, skin color, psoriasis, allergy, red area, general skin disorder or infection, tumor, sunburn, rash, scratch, pimple, acne, stria, insect bite, itch, bleeding, injury, inflammation, photodamage, pigmentation, tone, tattoo, at least one of percent burn and burn classification, mole, nevus, aspects of a skin lesion, such as structure, color, dimensions and asymmetry, melanoma, automated follow-up of pigmented skin lesions, dermally observed disorder, cutaneous lesion, cellulite, boil, blistering disease, congenital dermal syndrome, (sub)-cutaneous mycoses, melasma, vascular condition, rosacea, spider vein, texture, skin ulcer, wound healing, post-operative tracking, melanocytic lesion, non-melanocytic lesion, basal cell carcinoma, seborrhoic keratosis, sebum (oiliness), nail and hair-related concern, and the like, generating a normalized Red (R) and Blue (B) color channel histogram for each of the one or more images, correlating the normalized Red (R) and Blue (B) color channel histograms to a wavelength scale to obtain Red (R) and Blue (B) color channel spectral plots and convoluting the spectral plots by subtracting the spectral plot for the polarized optical electromagnetic signal from the non-polarized optical electromagnetic signal for each color to generate Red (R) and Blue (B) normalized, composite color channel spectral plots and subtracting the normalized, composite Blue (B) channel spectral plot from the normalized, composite Red (R) channel spectral plot thereby resulting in generation of a spectral signature for the skin.

Embodiments of the present disclose a method for managing sun exposure of skin. The method comprises in a pre-sun exposure phase, inputting the location information of a user, and other contextual ambient information therefor, thereby facilitating determination of the UV level thereof, determining a baseline information in connection with the skin in a part of the day from at least one of midnight and dawn to noon, scanning at least one most predictably susceptible zone of one or more zones of the skin predictably susceptible to sun exposure, determining the Sun Protection Factor (SPF) level in connection with at least one of a sunscreen and sunblock for use by the user based on one or more quantifiable qualitative and quantitative parameters therefor, recommending the determined SPF level for the at least one of the sunscreen and sunblock for use by the user thereby facilitating search, identification and selection of one or more of the at least one of the sunscreen and sunblock products, solutions and regimens by the user, in an in-sun exposure phase, determining whether or not the UV index of the location is confined in at least one of one or more predetermined ranges, and reminding the user to scan the skin at one or more predetermined intervals depending upon the confinement of the determined UV index in at least one of the one or more predetermined ranges.

Embodiments of the present invention disclose a method of managing treatment comprising capturing images of an organ, analyzing the captured images, determining at least one of susceptibility to at least one of a medical and non-medical condition and presence of the at least one of medical and non-medical condition, upon determination of the presence of at least one of medical and non-medical condition, measuring the level of severity of the at least one of medical and non-medical condition, categorizing the at least one of medical and non-medical condition based on the measured level of severity, recommending one or more of at least one of preventive, palliative, curative and cosmetic cum aesthetic, and at least one of new and enhanced personalized treatments based on the measured level of severity of the at least one of medical and non-medical condition, implementing the one or more of at least one of preventive, palliative, curative and cosmetic cum aesthetic, new and enhanced personalized treatments, tracking efficacy of the implemented one or more of at least one of preventive, palliative, curative and cosmetic cum aesthetic, new and enhanced personalized treatments and optimizing the one or more of at least one of preventive, palliative, curative and cosmetic cum aesthetic, new and enhanced personalized treatments based on the efficacies thereof.

Embodiments of the present invention disclose an opto-magnetic method for detecting at least one of a medical and non-medical condition of an organ. The method comprises in vivo detection by direct imaging using Opto-Magnetic Fingerprint (OMF) of the organ.

Embodiments of the present invention disclose a system for managing treatments. The system comprises a diagnosis subsystem. The diagnosis subsystem comprises an illumination subsystem for emitting polarized and unpolarized electromagnetic signals of multiple wavelengths, an imaging subsystem for capturing images of an organ illuminated with the polarized and unpolarized electromagnetic signals of multiple wavelengths, and a host computing subsystem. The host computing subsystem comprises an organ management module for analyzing the captured images. The organ management module comprises an organ risk assessment sub-module for determining susceptibility to at least one of a medical and non-medical condition of the organ, an organ state detection and prediction sub-module for determining the presence of the at least one of medical and non-medical condition, measuring the level of severity of the at least one of medical and non-medical condition, categorizing the at least one of medical and non-medical condition based on the measured level of severity, recommending one or more of at least one of preventive, palliative, curative and cosmetic cum aesthetic, and at least one of new and enhanced personalized treatments based on the measured level of severity of the at least one of medical and non-medical condition, implementing the one or more of at least one of preventive, palliative, curative and cosmetic cum aesthetic, new and enhanced personalized treatments, tracking efficacy of the implemented one or more of at least one of preventive, palliative, curative and cosmetic cum aesthetic, new and enhanced personalized treatments, and optimizing the one or more of at least one of preventive, palliative, curative and cosmetic cum aesthetic, new and enhanced personalized treatments based on the efficacies thereof.

These and other systems, methods, objects, features, and advantages of the present invention will be apparent to those skilled in the art from the following detailed description of the preferred embodiment and the drawings. All documents mentioned herein are hereby incorporated in their entirety by reference.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 depicts a block diagram of a system 100 for managing treatment, according to one or more embodiments;

FIG. 2 depicts a detailed block diagram of the host computing subsystem, of FIG. 1, comprising an organ management module, designed and implemented in accordance with one or more embodiments;

FIG. 3 is a flow diagram of a method of treating at least one of medical and non-medical condition, according to one or more embodiments; and

FIG. 4 depicts a computer system that is a computing device and can be utilized in various embodiments of the present invention, according to one or more embodiments.

While the method and apparatus is described herein by way of example for several embodiments and illustrative drawings, those skilled in the art will recognize that the method and system for managing treatments, is not limited to the embodiments or drawings described. It should be understood, that the drawings and detailed description thereto are not intended to limit embodiments to the particular form disclosed. Rather, the intention is to cover all modifications, equivalents and alternatives falling within the spirit and scope of the method and system for managing treatments defined by the appended claims. Any headings used herein are for organizational purposes only and are not meant to limit the scope of the description or the claims. As used herein, the word “may” is used in a permissive sense (i.e., meaning having the potential to), rather than the mandatory sense (i.e., meaning must). Similarly, the words “include”, “including”, and “includes” mean including, but not limited to.

### DETAILED DESCRIPTION

In certain embodiments, methods for managing treatment with enhanced qualitative and quantitative parameters and systems facilitating implementation of such methods are disclosed. Stated differently, in certain such embodiments, systems and apparatuses for practicing the principles of the invention are disclosed. More specifically, the system facilitates implementation of a method for managing treatment with enhanced qualitative and quantitative parameters. Still more specifically, the systems facilitates implementation of a method for managing treatment with enhanced qualitative and quantitative parameters, such as high economic feasibility, improved image stability, high adaptability, improved image capturability, reduced susceptibility to user or operator induced movements, increased power availability, improved ergonomics, improved operability, wired and wireless communicability, user selectable or automatic angular adjustability, user self image capturability, adaptive dynamic integrability or modularity, adaptive mountability, remote controllability, adaptive configurability, and the like.

Various embodiments of a method and system for managing treatment are described. In the following detailed description, numerous specific details are set forth to provide a thorough understanding of claimed subject matter. However, it will be understood by those skilled in the art that claimed subject matter may be practiced without these specific details. In other instances, methods, apparatuses or systems that would be known by one of ordinary skill have not been described in detail so as not to obscure claimed subject matter.

Some portions of the detailed description which follow are presented in terms of algorithms or symbolic representations of operations on binary digital signals stored within a memory of a specific apparatus or special purpose computing device or platform. In the context of this particular specification, the term specific apparatus or the like includes a general purpose computer once it is programmed to perform particular functions pursuant to instructions from program software. Algorithmic descriptions or symbolic representations are examples of techniques used by those of ordinary skill in the signal processing or related arts to convey the substance of their work to others skilled in the art. An algorithm is here, and is generally, considered to be a self-consistent sequence of operations or similar signal processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has proven convenient at times, principally for reasons of common usage, to refer to such signals as bits, data, values, elements, symbols, characters, terms, numbers, numerals or the like. It should be understood, however, that all of these or similar terms are to be associated with appropriate physical quantities and are merely convenient labels. Unless specifically stated otherwise, as apparent from the following discussion, it is appreciated that throughout this specification discussions utilizing terms such as “processing,” “computing,” “calculating,” “determining” or the like refer to actions or processes of a specific apparatus, such as a special purpose computer or a similar special purpose electronic computing device. In the context of this specification, therefore, a special purpose computer or a similar special purpose electronic computing device is capable of manipulating or transforming signals, typically represented as physical electronic or magnetic quantities within memories, registers, or other information storage devices, transmission devices, or display devices of the special purpose computer or similar special purpose electronic computing device.

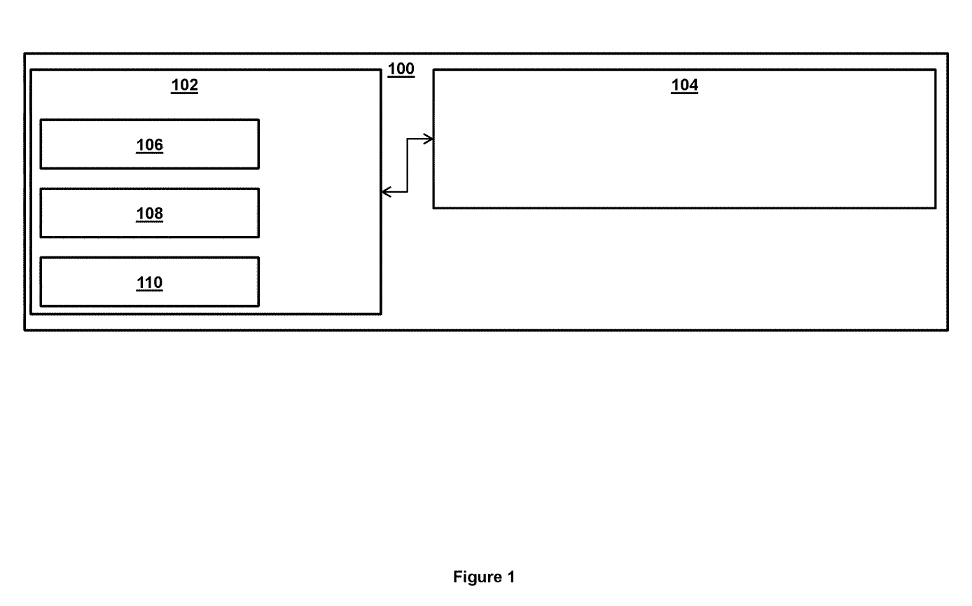

FIG. 1 depicts a block diagram of a system 100 for managing treatment, according to one or more embodiments.

The system 100 may comprise a diagnosis subsystem 102 and treatment subsystem 104. The diagnosis subsystem 102 may comprise an imaging (or sensor) subsystem 106, a host computing subsystem 108 and an illumination subsystem 110.

System 100, by virtue of its design and implementation, may facilitate execution of an Opto-Magnetic method based on interaction between electromagnetic radiation and matter, for instance light-matter interaction, via digital imaging for capturing images of organs, analyzing the captured images, profiling the human subjects based on the analyses, categorizing the human subjects based on the profiles, recommending at least one of treatments, therapies (or regimens) and health condition or care management procedures and tracking efficacy of the at least one of recommended treatments, therapies (or regimens) and health condition (or care management procedures) treatments thereby facilitating customization and optimization of treatments. Specifically, the Opto-Magnetic method may facilitate generation of unique spectral signatures (or Opto-Magnetic Fingerprint or OMF) from digitally captured images of the organs thereby facilitating analyzing the samples therefor, profiling the human subjects based on the analyses, categorizing the human subjects based on the profiles, recommending at least one of treatments, therapies (or regimens) and health condition (or care management procedures) and tracking efficacy of the at least one of recommended treatments, therapies (or regimens) and health condition (or care management procedures) treatments thereby facilitating customization and optimization of medical and non-medical treatments based on Opto-Magnetic properties of light-organ interaction.

The system 100 facilitates assessment of the status of organs and recommends therapies based on the assessment of the status and level of severity in connection with at least one of a medical and non-medical condition thereof. The system 100 also facilitates treatment of the organs. The system 100 facilitates measurement of severity of at least one of a medical and non-medical condition of the organs. The system 100 facilitates selection of an appropriate wavelength via utilization of the illumination subsystem 110. The system 100 facilitates selection of an appropriate treatment regimen based on the status of the organs.

The illumination subsystem 110 may be one or more electromagnetic radiation sources. In some embodiments, the Illumination subsystem 110 may be a set of light emitting diodes (LEDs).

The illumination subsystem 110 may be adapted to emit polarized and unpolarized electromagnetic signals of multiple wavelengths. The polarized electromagnetic signal is angled white light and unpolarized electromagnetic signal is non-angled white light.

As shown in FIG. 1, in some embodiments, the illumination subsystem 110 may be coupled to the sensor subsystem 106. In some other embodiments, the imaging (or sensor) subsystem 106 comprises the illumination unit 110. In some embodiments, the imaging (or sensor) subsystem 106 may be a portable handheld image scanner.

As depicted in FIG. 1, the sensor subsystem 106 may be a device that converts optical images (or optical signals) to electric signals. In some embodiments, the sensor subsystem 106 captures continuous digital images of organs. Specifically, the sensor subsystem 106 captures continuous digital images of the organs illuminated with white light both, non-angled and angled. By way of, and by no way of limitation, the sensor subsystem 106 may be anyone selected from a group consisting of a Complementary Metal-Oxide-Semiconductor (CMOS) image sensor, Charged Coupled Device (CCD) image sensor, and the like.

Again, as depicted in FIG. 1, the sensor subsystem 106 may be coupled to the host computing subsystem 108. In some embodiments, the system 100 is a single assembly of the imaging (or sensor) subsystem 106, host computing subsystem 108, illumination subsystem 110 and treatment subsystem 104.

The term “digital image” refers to a representation of a two-dimensional image using ones and zeros (or binary digits or bits). The digital image may be of vector or raster type depending on whether or not the image resolution is fixed. However, without qualifications the term “digital image” usually refers to raster images.

Likewise, the term “digital imaging or digital image acquisition” refers to creation of digital images, typically from a physical object. The term is often assumed to imply or include the processing, compression, storage, printing and display of such images.

Digital image processing is the use of computer algorithms to perform image processing on digital images. As a subfield of digital signal processing, digital image processing has many advantages over analog image processing; it allows a much wider range of algorithms to be applied to the input data, and can avoid problems such as the build-up of noise and signal distortion during processing.

For example, and in no way limiting the scope of the invention, in certain embodiments the sensor subsystem 106 may be selected on the basis of the following specifications: color is color or monochrome; optical format; horizontal pixels×vertical pixels; pixel size; one or more performance parameters, such as maximum frame rate, data rate, maximum power dissipation, quantum efficiency, dynamic range and supply voltage; output; one or more features, such as integrated Analog-to-Digital Converter (ADC) and micro lenses; and environment, such as operating temperature.

The term “image processing”, as used herein, refers to any form of signal processing for which the input is an image, such as photographs or frames of video. The output of image processing can be either an image or a set of characteristics or parameters related to the image. Most image-processing techniques involve treating the image as a two-dimensional signal and applying standard signal-processing techniques to it.

Image processing usually refers to digital image processing, but optical and analog image processing are also possible. The acquisition of images, i.e. producing the input image in the first place, is referred to as imaging.

The term “digital image processing”, as used herein, refers to the use of computer algorithms to perform image processing on digital images. As a subfield of digital signal processing, digital image processing has many advantages over analog image processing. For example, digital image processing allows a much wider range of algorithms to be applied to the input data and can avoid problems, such as the build-up of noise and signal distortion during processing.

Medical imaging refers to the techniques and processes used to create images of the human body (or parts thereof) for clinical purposes (medical procedures seeking to reveal, diagnose or examine disease) or medical science (including the study of normal anatomy and physiology).

As a discipline and in its widest sense, it is part of biological imaging and incorporates radiology (in the wider sense), radiological sciences, endoscopy, (medical) thermography, medical photography and microscopy (e.g. for human pathological investigations).

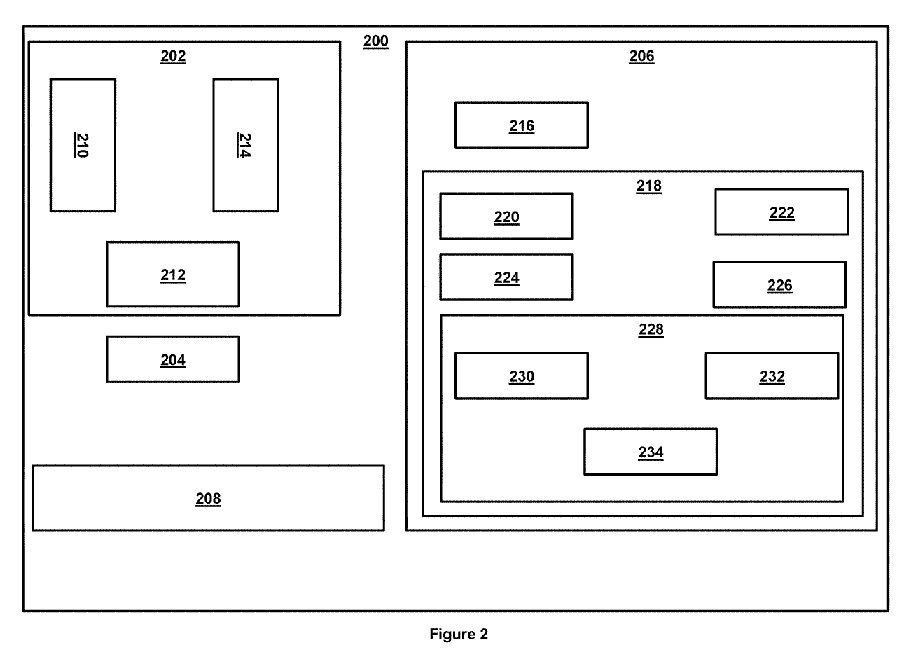

FIG. 2 depicts a detailed block diagram of the host computing subsystem, of FIG. 1, comprising an organ management module, designed and implemented in accordance with one or more embodiments.

The host computing subsystem 200 may comprise a processing unit 202, support circuits 204, a memory unit 206 and an Input/Output (or I/O) unit 208 respectively.

The host computing subsystem 200, by virtue of its design and implementation, facilitates performance of overall management of at least one of medical and non-medical condition in connection with an organ.

In some embodiments, the host computing subsystem 200 may be at least one of a portable computer, tablet computer, personal digital assistant (PDA), ultra mobile PC, Internet tablet, smartphone, carputer, pentop computer, and the like.

The processing unit 202 may comprise one or more commercially available microprocessors or microcontrollers that facilitate data processing and storage. The processing unit 202 may further comprise an Arithmetic Logic Unit (or ALU) 210, a Control Unit (or CU) 212 and a Register Unit (or RU) 214.

The support circuits 204 facilitate the operation of the processing unit 202 and include one or more clock circuits, power supplies, cache, input/output circuits, displays, and the like.

The memory unit 206 comprises at least one of Read Only Memory (ROM), Random Access Memory (RAM), disk drive storage, optical storage, removable storage and/or the like. The memory further 206 comprises an Operating System (OS) 216 and an organ management module 218.

The organ management module 218 comprises an organ risk assessment sub-module 220 and an organ state detection and prediction sub-module 222.

The organ risk assessment sub-module 220 facilitates analysis and assessment of overall susceptibility or propensity to at least one of a medical and non-medical condition in connection with the organ. Specifically, the organ risk assessment module 220 facilitates analysis and assessment of the susceptibility to risk (or risk of occurrence) of the at least one of medical and non-medical condition via computation of a risk number therefor.

As used herein, the term “medical or non-medical condition risk” refers to an overall risk or susceptibility of a person toward the at least one of medical and non-medical condition in connection with an organ. Medical or non-medical condition risk is principally based on healthcare, genetics, lifestyle and diet.

As used herein, the term “risk number” refers to a quantitative parameter based on one or more factors including, but not limited to, diet type, lifestyle and healthcare routines adhered to.

The term “diet” refers to a factor facilitating determination of a risk number or score of at least one of a medical and non-medical condition in connection with an organ based on a type of food consumed, for example, whether greasy or not, by a person and eating habits thereof.

The term “lifestyle” refers to a factor facilitating determination of a risk number or score of at least one of a medical and non-medical condition in connection with an organ based on the environmental exposure profile of a person based on the present and historical environmental exposure, for example in at least one of dirty, polluted and smoke-filled environment.

The term “healthcare routine” refers to a factor facilitating determination of a risk number or score of at least one of a medical and non-medical condition in connection with an organ based on healthcare routines or regimens adhered to by a person, for example frequency of washing the face, use of an appropriate product based on type of the skin of the person, for example certain products are counterproductive in acne thereby causing inflammation.

In operation, upon determination of the at least one of medical and non-medical condition risk based on a risk number, the organ risk assessment sub-module 220 facilitates reduction or minimization of the at least one of medical and non-medical condition risk upon performance of one or more specific actions by a person based on suggestions or recommendations provided by the organ risk assessment sub-module 220. Each of the one or more specific actions upon performance by a person results in reduction or minimization of the risk number or score.

The organ state detection and prediction sub-module 222 facilitates determination of the current actual state or status of an organ and prognosis of the likelihood of at least one of improvement and deterioration of the organ status based on the current actual status of the organ.

In operation, a user scans the organ using the portable handheld image scanner 106, of FIG. 1. For example, the portable handheld image scanner 106 may be an IPHONE®. In some embodiments, the host computing subsystem 200, of FIG. 2, may be coupled to the IPHONE®. The user selects organ and a portion thereof to scan based on a corresponding state of the organ. In some embodiments, the system 100 is a single assembly of the imaging (or sensor) subsystem 106, host computing subsystem 108, illumination subsystem 110 and treatment subsystem 104, all of the foregoing of FIG. 1.

In operation, using the system 100 a user learns about a scanned organ via achievement of the risk number or score for the scanned organ based on severity of the at least one of a medical and non-medical condition thereof on a measurement scale, for example ranging from a minimum of 0 (or best) to a maximum of 100 (or worst).

In some embodiments, using the system 100 a user learns about a scanned organ by achievement of a visual information (or confirmation) for presence of at least one of a medical and non-medical condition and a state thereof based on a color coding utilized to represent the state or severity of the at least one of medical and non-medical condition, for example a green color represents a best state, whereas a red color represents a worst state.

In some embodiments, using the system 100 a user learns about one or more characteristics of a scanned organ. For example, in some scenarios, a pore on the skin may be at least one of open and closed, wherein the open pore allows sebum to pass while the blocked pore retains previously stored matter therein. Likewise, in some other scenarios, glands may be at least one of active and inactive, wherein active glands are healthy. However, not healthy if the glands are hyperactive. For example, if a user's pores are blocked and the glands are active, the system 100 recommends or alerts the user to be cautious. Still however, if the user's pores are blocked and the glands are inactive, the system 100 recommends the user about short time to respond or to take necessary action. Still likewise, in some scenarios, an age of sebum may be at least young and old, wherein the older the sebum indicates the more is the probability of bacteria. In some scenarios, the area in at least one of vicinity and proximity of the scanned zit may be at least one inflamed and not inflamed.

In some embodiments, using the system 100 a user learns about the trends of a scanned organ via achievement of responses or answers to one or more queries in questionnaire posed therefor. For example, the user achieves responses or answers to one or more queries, such as “What is the scanned zit status trend or tendency over time?,” “Is the status of the scanned zit at least one of worsening and improving vis-à-vis a last scan of the zit?”

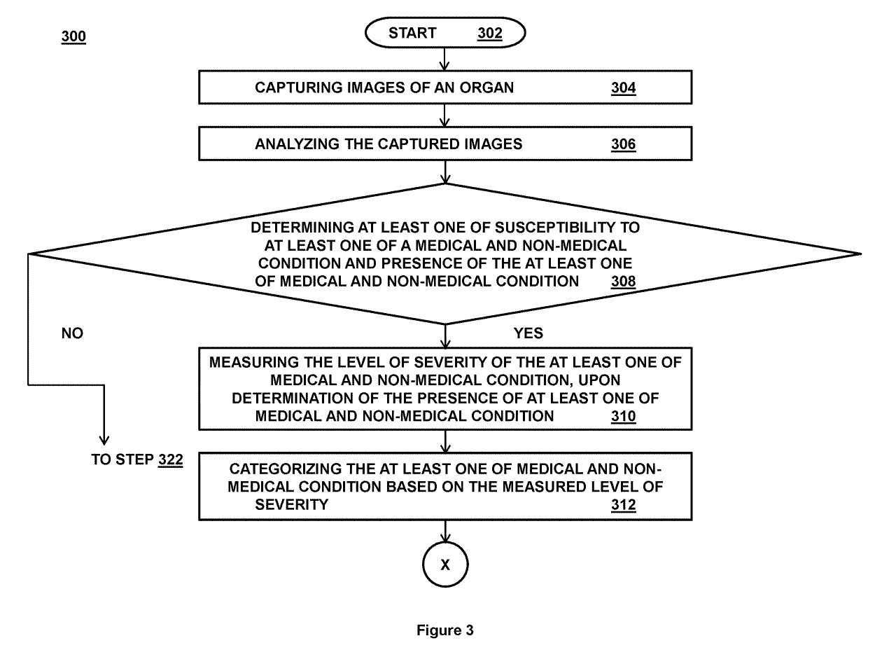

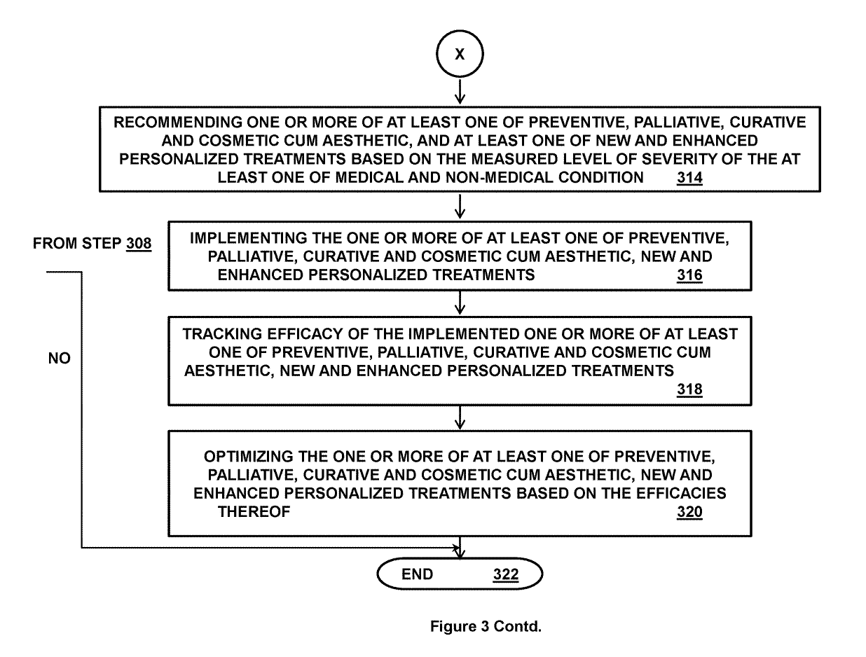

FIG. 3 is a flow diagram of a method of treating at least one of medical and non-medical condition, according to one or more embodiments.

The method 300 starts at step 302 and proceeds to step 304. At step 304, the method 300 comprises capturing images of an organ.

At step 306, the method 300 comprises analyzing the captured images.

At step 308, the method 300 comprises determining at least one of susceptibility to at least one of a medical and non-medical condition and presence of the at least one of medical and non-medical condition.

In the event that the at least one of medical and non-medical condition is detected in the organ, the method 300 proceeds to step 310. Else in the event that the at least one of medical and non-medical condition is not detected in the organ, the method 300 proceeds to step 322.

At step 310, the method 300 comprises measuring the level of severity of the at least one of medical and non-medical condition, upon determination of the presence of at least one of medical and non-medical condition.

At step 312, the method 300 comprises categorizing the at least one of medical and non-medical condition based on the measured level of severity.

At step 314, the method 300 comprises recommending one or more of at least one of preventive, palliative, curative and cosmetic cum aesthetic, and at least one of new and enhanced personalized treatments based on the measured level of severity of the at least one of medical and non-medical condition.

At step 316, the method 300 comprises implementing the one or more of at least one of preventive, palliative, curative and cosmetic cum aesthetic, new and enhanced personalized treatments.

At step 318, the method 300 comprises tracking efficacy of the implemented one or more of at least one of preventive, palliative, curative and cosmetic cum aesthetic, new and enhanced personalized treatments.

At step 320, the method 300 comprises optimizing the one or more of at least one of preventive, palliative, curative and cosmetic cum aesthetic, new and enhanced personalized treatments based on the efficacies thereof.

The method 300 proceeds to step 322 and ends.

In some embodiments, the organ management module for analysis of an organ subjected to risk assessment in connection with at least one of a medical and non-medical condition of the organ and current state detection and future state prediction thereof comprises generation of unique spectral signatures from the digitally captured images of the organ, in accordance with the principles of the invention. Specifically, in such embodiments, the organ management module utilizes the continuously captured digital images of the organ illuminated with white light both, non-angled and angled. More specifically, the organ management module takes into consideration the digital images in Red (R), Green (G) and Blue (B) (or RGB) system for purposes of analysis.

Further, as shown in FIG. 2, the organ management module 218 comprises a Fourier transform sub-module 224, a spectral analyzer sub-module 226 and an Opto-Magnetic Fingerprint Generator (or OMFG) sub-module 228, respectively.

In certain embodiments, the Fourier transform sub-module 224 is in essence a Discrete-Time Fourier Transform (or DTFT).

The term “DTFT”, as used herein, refers to one of the specific forms of Fourier analysis. As such, it transforms one function into another, which is called the frequency domain representation, or simply the “DTFT”, of the original function, which is often a function in the time-domain. But, the DTFT requires an input function that is discrete.

Such inputs are often created by sampling a continuous function, like a person's voice. The DTFT frequency-domain representation is always a periodic function. Since one period of the function contains all of the unique information, it is sometimes convenient to say that the DTFT is a transform to a “finite” frequency-domain (the length of one period), rather than to the entire real line.

DTFT 224 converts time-domain digital signals into corresponding frequency-domain digital signals.

DTFT 224 is coupled to the spectrum analyzer sub-module 226.

As used herein, the term “spectrum analyzer” refers to a device used to examine the spectral composition of some electrical, acoustic, or optical waveform. It may also measure the power spectrum. In general, there are three types of spectrum analyzers, such as analog, digital and real-time spectrum analyzers. Firstly, an analog spectrum analyzer uses either a variable band-pass filter whose mid-frequency is automatically tuned (i.e. shifted, swept) through the range of frequencies of the spectrum to be measured or a superheterodyne receiver, wherein the local oscillator is swept through a range of frequencies. Secondly, a digital spectrum analyzer computes the Discrete Fourier transform (or DFT), a mathematical process that transforms a waveform into the components of its frequency spectrum. Eventually, some spectrum analyzers, such as “real-time spectrum analyzers”, use a hybrid technique where the incoming signal is first down-converted to a lower frequency using superheterodyne techniques and then analyzed using fast Fourier transformation (FFT) techniques.

In some embodiments, the spectrum (or spectral) analyzer sub-module facilitating analysis of the organ subjected to at least one of medical and non-medical condition risk assessment and state detection and prediction thereof is disclosed. Specifically, the spectrum (or spectral) analyzer sub-module takes into consideration digital images of the samples in Red (R), Green (G) and Blue (B) (or RGB) system in order to analyze the organ. In certain such embodiments, basic pixel data in Red (R) and Blue (B) channels for both white diffuse light (or W) and reflected polarized light (or P) is selected. In here, the algorithm for data analysis is based on chromaticity diagram called “Maxwell's triangle” and spectral convolution.

In some embodiments, the digital images in Red (R), Green (G) and Blue (B) (or RGB) system are taken into consideration for purposes of spectral analysis. Specifically, basic pixel data in Red (R) and Blue (B) channels for white diffuse light (or W) and reflected polarized white light (or P) is selected. More specifically, the algorithm for data analysis is based on chromaticity diagram called “Maxwell's triangle” and spectral convolution operation, in accordance with a ratio of (R−B) & (W−P). Noticeably, the abbreviated designation implies that Red (R) minus Blue (B) wavelength of White light (W) and reflected Polarized light (P) are used in spectral convolution algorithm to calculate data for Opto-Magnetic Fingerprint (OMF) of matter both, organic and inorganic. Consequently, method and algorithm for creating unique spectral fingerprint are based on the convolution of RGB color channel spectral plots generated from digital images that capture single and multi-wavelength light-matter interaction for different paramagnetic materials, such as Al, Mn and Ti, diamagnetic materials, such as Cu, C and Zn, alloys, such asPb1-xMnxTe, Biomolecules and biological tissues as paramagnetic/diamagnetic materials, such as skin, biological water, amniotic fluid, blood plasma and the like.

Further, incident white light can give different information about properties of thin layer of matter, such as skin acne, depending on the angle of light incidence. In use, when the incident white light diffuse, the reflected white light is composed of electrical and magnetic components, wherein diffuse incident light inclined under certain angle will produce reflected light, which contains only electrical component of light.

As shown in FIG. 2, the spectrum analyzer sub-module 226 may be coupled to the OMFG sub-module 228.

The OMFG sub-module 228 comprises a color histogram generator unit 230, a spectral plot generator unit 232 and a convolution unit 234.

The OMFG sub-module 228, by virtue of its design and implementation, facilitates generation of unique spectral signatures from digitally captured images of organs. Specifically, the generated spectral signatures of the organs facilitate detection of at least one of a medical and non-medical condition of the organs based on Opto-Magnetic properties of light-organ interaction.

The color histogram generator unit 230, by virtue of its design, generates a normalized Red (R) and Blue (B) color channel histogram for each of the one or more images of the organs.

The term “color histogram”, as used in computer graphics and photography, refers to is a representation of the distribution of colors in an image, derived by counting the number of pixels of each of given set of color ranges in a typically two-dimensional (2D) or three-dimensional (3D) color space. A histogram is a standard statistical description of a distribution in terms of occurrence frequencies of different event classes; for color, the event classes are regions in color space. An image histogram of scalar pixel values is more commonly used in image processing than is a color histogram. The term “image histogram” refers to a type of histogram which acts as a graphical representation of the tonal distribution in a digital image. It plots the number of pixels for each tonal value. By looking at the histogram for a specific image a viewer is able to judge the entire tonal distribution at a glance.

Typically, color histograms are flexible constructs that can be built from images in various color spaces, whether RGB, rg chromaticity or any other color space of any dimension. A histogram of an image is produced first by discretization of the colors in the image into a number of bins, and counting the number of image pixels in each bin. For example, a Red-Blue chromaticity histogram can be formed by first normalizing color pixel values by dividing RGB values by R+G+B, then quantizing the normalized R and B coordinates into N bins each, where N=4, which might yield a 2D histogram that looks like this table:

Table 1 exhibits a tabular representation in connection with a 2D Red-Blue chromaticity histogram generated by first normalizing color pixel values by dividing RGB values by R+G+B, then quantizing the normalized R and B coordinates into N bins each, where N=4.

| | | R | | | |
|---|---|---|---|---|---|
| B | 0-63 | 43 | 78 | 18 | 0 |
| | 64-127 | 45 | 67 | 33 | 2 |
| | 128-191 | 127 | 58 | 25 | 8 |
| | 192-255 | 140 | 47 | 47 | 13 |

As shown in FIG. 2, the color histogram generator unit 230 may be coupled to the spectral plot generator unit 232.

Spectral plot generator unit 232 generates Red (R) and Blue (B) color channel spectral plots by correlating the normalized Red (R) and Blue (B) color channel histograms to a wavelength scale. In certain embodiments, a unit scale on the spectral signature is a difference of wavelength.

In general, color digital images are made of pixels and, in turn, pixels are made of combinations of primary colors. As used in the current context, the term “channel” refers to the grayscale image of the same size as a color image, made of just one of these primary colors. For instance, an image from a standard digital camera will have a red, green and blue channel. A grayscale image has just one channel. Further, an RGB image has three channels, namely Red (R), Green (G) and Blue (B). For example, if the RGB image is 24-bit then each channel has 8 bits, for R, G and B. Stated differently, the image is composed of three grayscale images, where each grayscale image can store discrete pixels with conventional brightness intensities between 0 and 255. Whereas, if the RGB image is 48-bit (i.e. very high resolution), each channel is made of 16-bit grayscale images.

The periodogram is an estimate of the spectral density of a signal. The term “spectral plot” refers to a smoothed version of the periodogram. Smoothing is performed to reduce the effect of measurement noise.

The convolution unit 234 convolutes the Red (R) and Blue (B) color channel spectral plots by subtracting the spectral plot for the polarized optical electromagnetic signal from the non-polarized optical electromagnetic signal for each color to generate Red (R) and Blue (B) normalized, composite color channel spectral plots and subtracting the normalized, composite Blue (B) channel spectral plot from the normalized, composite Red (R) channel spectral plot thereby resulting in generation of a spectral signature for the organ.

In some embodiments, the spectral signature is analyzed for at least one of number of crests and troughs, amplitude, shape of peaks, intermediate structures and patterns. In some embodiments, the spectral signature is analyzed for material composition, identification, purity and the like.

The embodiments of the present invention may be embodied as methods, system, apparatus, electronic devices, and/or computer program products. Accordingly, the embodiments of the present invention may be embodied in hardware and/or in software (including firmware, resident software, micro-code, etc.), which may be generally referred to herein as a “circuit” or “module”. Furthermore, the present invention may take the form of a computer program product on a computer-usable or computer-readable storage medium having computer-usable or computer-readable program code embodied in the medium for use by or in connection with an instruction execution system. In the context of this document, a computer-usable or computer-readable medium may be any medium that can contain, store, communicate, propagate, or transport the program for use by or in connection with the instruction execution system, apparatus, or device. These computer program instructions may also be stored in a computer-usable or computer-readable memory that may direct a computer or other programmable data processing apparatus to function in a particular manner, such that the instructions stored in the computer usable or computer-readable memory produce an article of manufacture including instructions that implement the function specified in the flowchart and/or block diagram block or blocks.

The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium include the following: hard disks, optical storage devices, a transmission media such as those supporting the Internet or an intranet, magnetic storage devices, an electrical connection having one or more wires, a portable computer diskette, a random access memory (RAM), a read-only memory (ROM), an erasable programmable read-only memory (EPROM or Flash memory), an optical fiber, and a compact disc read-only memory (CD-ROM).

Computer program code for carrying out operations of the present invention may be written in an object oriented programming language, such as Java®, Smalltalk or C++, and the like. However, the computer program code for carrying out operations of the present invention may also be written in conventional procedural programming languages, such as the “C” programming language and/or any other lower level assembler languages. It will be further appreciated that the functionality of any or all of the program modules may also be implemented using discrete hardware components, one or more Application Specific Integrated Circuits (ASICs), or programmed Digital Signal Processors or microcontrollers.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the present disclosure and its practical applications, to thereby enable others skilled in the art to best utilize the invention and various embodiments with various modifications as may be suited to the particular use contemplated.

### Example Computer System

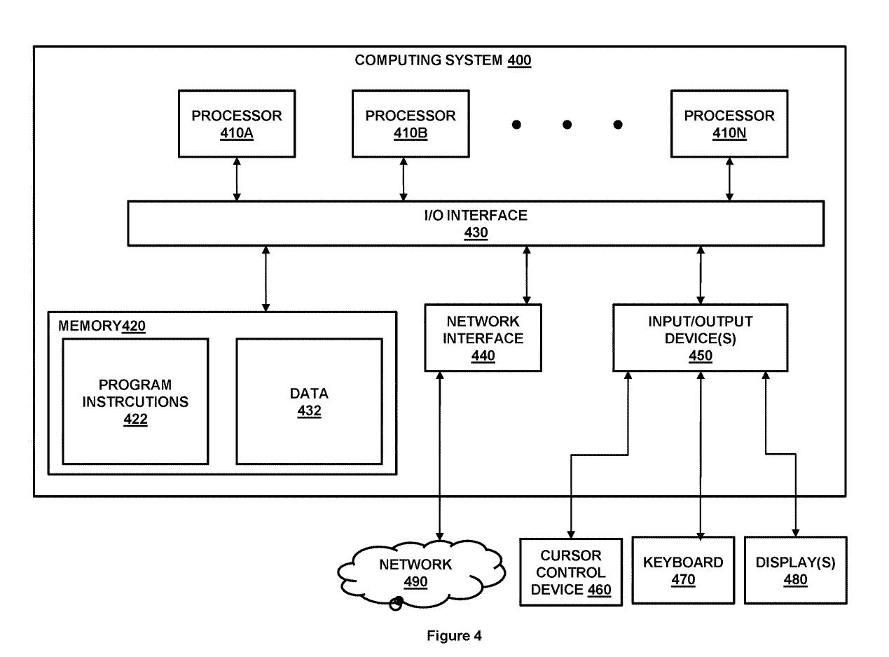

FIG. 4 depicts a computer system that is a computing device and can be utilized in various embodiments of the present invention, according to one or more embodiments.

Various embodiments of method and apparatus for managing skin health, as described herein, may be executed on one or more computer systems, which may interact with various other devices. One such computer system is computer system 400 illustrated by FIG. 4, which may in various embodiments implement any of the elements or functionality illustrated in FIGS. 1-3. In various embodiments, computer system 400 may be configured to implement methods described above. The computer system 400 may be used to implement any other system, device, element, functionality or method of the above-described embodiments. In the illustrated embodiments, computer system 400 may be configured to implement method 300, as processor-executable executable program instructions 422 (e.g., program instructions executable by processor(s) 410A-N) in various embodiments.

In the illustrated embodiment, computer system 400 includes one or more processors 410A-N coupled to a system memory 420 via an input/output (I/O) interface 430. The computer system 400 further includes a network interface 440 coupled to I/O interface 430, and one or more input/output devices 450, such as cursor control device 460, keyboard 470, and display(s) 480. In various embodiments, any of components may be utilized by the system to receive user input described above. In various embodiments, a user interface (e.g., user interface) may be generated and displayed on display 480. In some cases, it is contemplated that embodiments may be implemented using a single instance of computer system 400, while in other embodiments multiple such systems, or multiple nodes making up computer system 400, may be configured to host different portions or instances of various embodiments. For example, in one embodiment some elements may be implemented via one or more nodes of computer system 400 that are distinct from those nodes implementing other elements. In another example, multiple nodes may implement computer system 400 in a distributed manner.

In different embodiments, computer system 400 may be any of various types of devices, including, but not limited to, a personal computer system, desktop computer, laptop, notebook, or netbook computer, mainframe computer system, handheld computer, workstation, network computer, a camera, a set top box, a mobile device, a consumer device, video game console, handheld video game device, application server, storage device, a peripheral device such as a switch, modem, router, or in general any type of computing or electronic device.

In various embodiments, computer system 400 may be a uniprocessor system including one processor 410, or a multiprocessor system including several processors 410 (e.g., two, four, eight, or another suitable number). Processors 410A-N may be any suitable processor capable of executing instructions. For example, in various embodiments processors 410 may be general-purpose or embedded processors implementing any of a variety of instruction set architectures (ISAs), such as the x96, POWERPC®, SPARC®, or MIPS® ISAs, or any other suitable ISA. In multiprocessor systems, each of processors 410a-n may commonly, but not necessarily, implement the same ISA.

System memory 420 may be configured to store program instructions 422 and/or data 432 accessible by processor 410. In various embodiments, system memory 420 may be implemented using any suitable memory technology, such as static random access memory (SRAM), synchronous dynamic RAM (SDRAM), nonvolatile/Flash-type memory, or any other type of memory. In the illustrated embodiment, program instructions and data implementing any of the elements of the embodiments described above may be stored within system memory 420. In other embodiments, program instructions and/or data may be received, sent or stored upon different types of computer-accessible media or on similar media separate from system memory 420 or computer system 400.

In one embodiment, I/O interface 430 may be configured to coordinate I/O traffic between processor 410, system memory 420, and any peripheral devices in the device, including network interface 440 or other peripheral interfaces, such as input/output devices 450. In some embodiments, I/O interface 430 may perform any necessary protocol, timing or other data transformations to convert data signals from one components (e.g., system memory 420) into a format suitable for use by another component (e.g., processor 410). In some embodiments, I/O interface 430 may include support for devices attached through various types of peripheral buses, such as a variant of the Peripheral Component Interconnect (PCI) bus standard or the Universal Serial Bus (USB) standard, for example. In some embodiments, the function of I/O interface 430 may be split into two or more separate components, such as a north bridge and a south bridge, for example. Also, in some embodiments some or all of the functionality of I/O interface 430, such as an interface to system memory 420, may be incorporated directly into processor 410.

Network interface 440 may be configured to allow data to be exchanged between computer system 400 and other devices attached to a network (e.g., network 490), such as one or more external systems or between nodes of computer system 400. In various embodiments, network 490 may include one or more networks including but not limited to Local Area Networks (LANs) (e.g., an Ethernet or corporate network), Wide Area Networks (WANs) (e.g., the Internet), wireless data networks, some other electronic data network, or some combination thereof. In various embodiments, network interface 440 may support communication via wired or wireless general data networks, such as any suitable type of Ethernet network, for example; via telecommunications/telephony networks such as analog voice networks or digital fiber communications networks; via storage area networks such as Fiber Channel SANs, or via any other suitable type of network and/or protocol.

Input/output devices 450 may, in some embodiments, include one or more display terminals, keyboards, keypads, touchpads, scanning devices, voice or optical recognition devices, or any other devices suitable for entering or accessing data by one or more computer systems 400. Multiple input/output devices 450 may be present in computer system 400 or may be distributed on various nodes of computer system 400. In some embodiments, similar input/output devices may be separate from computer system 400 and may interact with one or more nodes of computer system 400 through a wired or wireless connection, such as over network interface 440.

In some embodiments, the illustrated computer system may implement any of the methods described above, such as the method illustrated by the flowchart of FIG. 3. In other embodiments, different elements and data may be included.

Those skilled in the art will appreciate that computer system 400 is merely illustrative and is not intended to limit the scope of embodiments. In particular, the computer system and devices may include any combination of hardware or software that can perform the indicated functions of various embodiments, including computers, network devices, Internet appliances, PDAs, wireless phones, pagers, etc. Computer system 400 may also be connected to other devices that are not illustrated, or instead may operate as a stand-alone system. In addition, the functionality provided by the illustrated components may in some embodiments be combined in fewer components or distributed in additional components. Similarly, in some embodiments, the functionality of some of the illustrated components may not be provided and/or other additional functionality may be available.

Those skilled in the art will also appreciate that, while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computer system 400 may be transmitted to computer system 400 via transmission media or signals such as electrical, electromagnetic, or digital signals, conveyed via a communication medium such as a network and/or a wireless link. Various embodiments may further include receiving, sending or storing instructions and/or data implemented in accordance with the foregoing description upon a computer-accessible medium or via a communication medium. In general, a computer-accessible medium may include a storage medium or memory medium such as magnetic or optical media, e.g., disk or DVD/CD-ROM, volatile or non-volatile media such as RAM (e.g., SDRAM, DDR, RDRAM, SRAM, etc.), ROM, etc.

The methods described herein may be implemented in software, hardware, or a combination thereof, in different embodiments. In addition, the order of methods may be changed, and various elements may be added, reordered, combined, omitted, modified, etc. All examples described herein are presented in a non-limiting manner. Various modifications and changes may be made as would be obvious to a person skilled in the art having benefit of this disclosure. Realizations in accordance with embodiments have been described in the context of particular embodiments. These embodiments are meant to be illustrative and not limiting. Many variations, modifications, additions, and improvements are possible. Accordingly, plural instances may be provided for components described herein as a single instance. Boundaries between various components, operations and data stores are somewhat arbitrary, and particular operations are illustrated in the context of specific illustrative configurations. Other allocations of functionality are envisioned and may fall within the scope of claims that follow. Finally, structures and functionality presented as discrete components in the example configurations may be implemented as a combined structure or component. These and other variations, modifications, additions, and improvements may fall within the scope of embodiments as defined in the claims that follow.

While the foregoing is directed to embodiments of the present invention, other and further embodiments of the invention may be devised without departing from the basic scope thereof, and the scope thereof is determined by the claims that follow.

1. A method for generating a spectral signature of a material, the method comprising:
capturing one or more images of the material illuminated with non-polarized (W) and reflected polarized (P) optical electromagnetic signals;
generating a normalized Red (R) and Blue (B) color channel histogram for each of the one or more images;
correlating the normalized Red (R) and Blue (B) color channel histograms to a wavelength scale to obtain Red (R) and Blue (B) color channel spectral plots; and
convoluting the spectral plots by subtracting the spectral plot for the polarized optical electromagnetic signal from the non-polarized optical electromagnetic signal for each color to generate Red (R) and Blue (B) normalized, composite color channel spectral plots and subtracting the normalized, composite Blue (B) channel spectral plot from the normalized, composite Red (R) channel spectral plot thereby resulting in generation of a spectral signature for the material.

2. A method of managing treatment comprising:
capturing images of an organ;
analyzing the captured images;
determining at least one of susceptibility to at least one of a medical and non-medical condition and presence of the at least one of medical and non-medical condition;
upon determination of the presence of at least one of medical and non-medical condition, measuring the level of severity of the at least one of medical and non-medical condition;
categorizing the at least one of medical and non-medical condition based on the measured level of severity;
recommending one or more of at least one of preventive, palliative, curative and cosmetic cum aesthetic, and at least one of new and enhanced personalized treatments based on the measured level of severity of the at least one of medical and non-medical condition;
implementing the one or more of at least one of preventive, palliative, curative and cosmetic cum aesthetic, new and enhanced personalized treatments;
tracking efficacy of the implemented one or more of at least one of preventive, palliative, curative and cosmetic cum aesthetic, new and enhanced personalized treatments; and
optimizing the one or more of at least one of preventive, palliative, curative and cosmetic cum aesthetic, new and enhanced personalized treatments based on the efficacies thereof.

3. A system for managing treatments, the apparatus comprising:
a diagnosis subsystem comprising:
an illumination subsystem for emitting polarized and unpolarized electromagnetic signals of multiple wavelengths, and
an imaging subsystem for capturing images of an organ illuminated with the polarized and unpolarized electromagnetic signals of multiple wavelengths; and
a host computing subsystem comprising:
an organ management module for analyzing the captured images comprising:
an organ risk assessment sub-module for determining susceptibility to at least one of a medical and non-medical condition of the organ, and
an organ state detection and prediction sub-module for determining the presence of the at least one of medical and non-medical condition, measuring the level of severity of the at least one of medical and non-medical condition, categorizing the at least one of medical and non-medical condition based on the measured level of severity, recommending one or more of at least one of preventive, palliative, curative and cosmetic cum aesthetic, and at least one of new and enhanced personalized treatments based on the measured level of severity of the at least one of medical and non-medical condition, implementing the one or more of at least one of preventive, palliative, curative and cosmetic cum aesthetic, new and enhanced personalized treatments, tracking efficacy of the implemented one or more of at least one of preventive, palliative, curative and cosmetic cum aesthetic, new and enhanced personalized treatments, and optimizing the one or more of at least one of preventive, palliative, curative and cosmetic cum aesthetic, new and enhanced personalized treatments based on the efficacies thereof.

4. The apparatus of claim 3, wherein the illumination and imaging subsystems comprise a single assembly removably coupled to the host computing subsystem.

5. The apparatus of claim 4, the electromagnetic signals of multiple wavelengths facilitate treatments.
